(19) United States
(12) Patent Application Publication
(10) Pub. No.: US 2009/0060859 A1
(43) Pub. Date: Mar. 5, 2009

(54) IMIDAZOLIUM ALKYL (METH)ACRYLATE POLYMERS

(75) Inventors: Ivette Garcia Castro, Ludwigshafen (DE); Yvonne Dieckmann, Hassloch (DE); Ingrid Haupt, Frankenthal (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/201,941

(22) Filed: Aug. 29, 2008

(30) Foreign Application Priority Data
Sep. 5, 2007 (EP) 07115757.2

(51) Int. Cl. A61K 8/81; C08F 26/06; A61Q 5/00

(52) U.S. Cl. 424/70.15; 526/263; 424/78.02

(57) ABSTRACT

The present invention relates to the use of imidazolium alkyl(meth)acrylate homo- and copolymers in cosmetic preparations and to the corresponding cosmetic preparations.

The present invention relates to the use of imidazolium alkyl(meth)acrylate homo- and copolymers in cosmetic preparations and to the corresponding cosmetic preparations.

N-Vinylimidazole polymers are used for highly diverse purposes in cosmetic preparations, as color transfer inhibitors, for the treatment of surfaces and in the electronics industry. Here, the imidazole radical assumes numerous functions, such as, for example, complexation and increasing the solubility. The low tendency of N-vinylimidazole to copolymerize with other monomers sometimes presents difficulties in the production of the N-vinylimidazole polymers.

WO 2008/026064 describes polymers which are produced starting from ionic liquids. It is the aim to permit controlled adsorption and desorption of CO₂, which exhibits a high affinity to polymers of ionic liquids. Polymers based on 1-[2-(methacryloyloxy)ethyl]-3-butylimidazolium tetrafluoroborate are described for this purpose.

Wagner et al. (Research Disclosure No. 24315, July 1984) describe the production of 2-(1-imidazolyl)ethyl methacrylate (referred to below as ImEMA), its homopolymer and the copolymerization with methyl methacrylate, and also the quaternization of the polymers. Use in mordants for photography is described.

Simmons et al. (Macromolecules 1998, 31, 9075-8077) describe the production of ImEMA, its homopolymerization and copolymerization with dimethylaminoethyl methacrylate (DMAEMA) to give diblock and random copolymers. The enzyme-analogous use of these polymers for hydrolysis is described.

Simmons et al. (J. Polym. Sci. A 1999, 37, 1501-1512) describe the production of ImEMA and its oligomerization and homopolymerization. The hydrolytic catalysis activity of these compounds is investigated,

Patrickios et al. (Colloids & Surfaces A 2000, 167, 61-72) describe linear and crosslinked ImEMA homopolymers and copolymers with DMAEMA or methyl methacrylate (MMA). Also investigated here is the hydrolytic catalysis activity of these compounds.

Hadjikallis et al. (Polymer 2002, 43, 7269-7273) describe the production of ImEMA-tetrahydropyranyl methacrylate block copolymers and their hydrolysis to give amphiphilic ImEMA-methacrylic acid block copolymers.

It was an object of the present invention to provide cationic or cationogenic polymers which can be produced either with high or with low molecular weights in a simple way and have advantageous properties when used in cosmetics.

This object was achieved through the use of polymers which comprise, in copolymerized form, at least one compound of the general formula (I) and/or its cationic form (Ia)

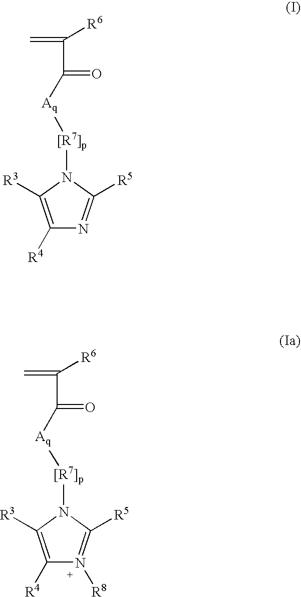

where

R³, R⁴ and R⁵, independently of one another, are H or C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₃-C₁₅-cycloalkylcarbonyl, aryl, arylcarbonyl, a heterocycle or a halogen atom,

R⁶ is H or methyl,

R⁷ is a divalent organic radical, such as, for example, alkylene or arylene,

R⁸ is H or C₁C₁₀-alkyl,

A is O or NH,

p, q independently of one another are 0 or 1,

in cosmetic preparations, in particular, the cosmetic preparations are haircare preparations.

In a preferred embodiment of the invention, p and q are not 0 at the same time.

Preferably, the compounds (I) and (Ia) are (meth)acrylic acid esters of compounds of the following general formula (II)

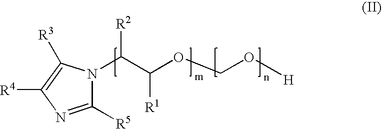

in which

R¹ and R², independently of one another, may be hydrogen or C₁-C₂₀-alkyl,

R³, R⁴ and R⁵, independently of one another, may be hydrogen or C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₃-C₁₅-cycloalkylcarbonyl, aryl, arylcarbonyl, a heterocycle or a halogen atom, and

m and n are in each case integers in the range from 0 to 20, where m and n cannot be 0 at the same time,

and in which those unite which are bracketed by the variables m and n are present in any desired order,

and in, which, when m≧2, the radicals R¹ and R² are in each case independent of one another in the respective units.

The term “polymers to be used according to the invention” below stands for all polymers which comprise compounds of the formula (I) and/or (Ia) in copolymerized form.

These may be random, gradient, block or graft copolymers, which may be linear or branched.

Specifically, the various radicals R in the stated collective terms have the following meaning:

C₁-C₂₀-Alkyl: straight-chain or branched hydrocarbon radicals having up to 20 carbon atoms, preferably C₁-C₁₀-alkyl, such as methyl, ethyl, propyl, isopropyl, n-butyl, sec-butyl, tert-butyl, 1,1-dimethylethyl, pentyl, 2-methylbutyl, 1,1-dimethylpropyl, 1,2-dimethylpropyl, 2,2-dimethylpropyl, 1-ethylpropyl, hexyl, 2-methylpentyl, 3-methylpentyl, 1,1-dimethylbutyl, 1,2-dimethylbutyl, 1,3-dimethylbutyl, 2,2-dimethyl-butyl, 2,3-dimethylbutyl, 3,3-dimethylbutyl, 2-ethylbutyl, 1,1,2-trimethylpropyl, 1,2,2-trimethylpropyl, 1-ethyl-methylpropyl, 1-ethyl-2-methylpropyl, heptyl, octyl, 2-ethylhexyl, 2,4,4-trimethylpentyl, 1,1,3,3-tetramethylbutyl, nonyl and decyl, and isomers thereof.

C₁-C₂₀-Alkylcarbonyl: a straight-chain or branched alkyl group having 1 to 20 carbon atoms (as specified above), which is bonded on via a carbonyl group (—CO—), preferably C₁-C₁₀-alkylcarbonyl such as, for example, formyl, acetyl, n- or isopropionyl, n-, iso-, sec- or tert-butanoyl, n-iso-, sec- or tert-pentanoyl, n- or isononanoyl, n-dodecanoyl, C₂-C₂₀-Alkenyl; unsaturated, straight-chain or branched hydrocarbon radicals having 2 to 20 carbon atoms and a double bond in any desired position, preferably C₂-C₁₀-alkenyl, such as ethenyl, 1-propenyl, 2-propenyl, 1-methylethenyl, 1-butenyl, 2-butenyl, 3-butenyl, 1-methyl-1-propenyl, 2-methyl-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-butenyl, 3-methyl-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl; and the isomers of heptenyl, octenyl, nonenyl and decenyl.

C₂-C₂₀-Alkenylcarbonyl: unsaturated, straight-chain or branched hydrocarbon radicals having 2 to 20 carbon atoms and a double bond in any desired position (as specified above), which are bonded on via a carbonyl group (—CO—), preferably C₂-C₁₀-alkylcarbonyl, such as, for example, ethenoyl, propenoyl, butenoyl, pentenoyl, nonenoyl and isomers thereof.

C₂-C₂₀-Alkynyl: straight-chain or branched hydrocarbon groups having 2 to 20 carbon atoms and a triple bond in any desired position, preferably C₂-C₁₀-alkynyl such as ethynyl 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl, and the isomers of heptynyl, octynyl, nonynyl, decynyl, C₂₀-C₂₀-Alkynylcarbonyl: unsaturated, straight-chain or branched hydrocarbon radicals having 2 to 20 carbon atoms and a triple bond in any desired position (as specified above) which are bonded on via a carbonyl group (—CO—), preferably C₂-C₁₀-alkynyl-carbonyl, such as, for example, propynoyl, butynoyl, pentynoyl, nonynoyl, decynoyl, and isomers thereof.

C₃-C₁₅-Cycloalkyl: monocyclic, saturated hydrocarbon groups having 3 to 15 carbon ring members, preferably C₃-C₈-cycloalkyl, such as cyclopropyl, cyclobutyl, cyclopentyl, cyclohexyl, cycloheptyl and cyclooctyl, and a saturated or unsaturated cyclic system, such as, for example norbornyl or norbenyl.

C₅C₁₅-Cycloalkylcarbonyl: monocyclic, saturated hydrocarbon groups having 3 to 15 carbon ring members (as specified above), which are on via a carbonyl group (—CO—), preferably C₃-C₈-cycloalkylcarbonyl.

Aryl: a mono- to trinuclear aromatic ring system comprising 6 to 14 carbon ring members, e.g. phenyl, naphthyl and anthracenyl, preferably a mono- to binuclear, particularly preferably a mononuclear, aromatic ring system,

Arylcarbonyl: preferably a mono- to trinuclear aromatic ring system (as specified above), which is bonded on via a carbonyl group (—CO—), such as, for example, benzoyl, preferably a mono- to binuclear, particularly preferably a mononuclear, aromatic ring system.

Heterocycles: five- to twelve-membered, preferably five to nine-membered, particularly preferably five- to six-membered ring system having oxygen, nitrogen and/or sulfur atoms and, if appropriate, having a plurality of rings, such as, for example, furyl, thiophenyl, pyrryl, pyridyl, indolyl, benzoxazolyl, dioxolyl, dioxyl, benzimidazolyl, benzthiazolyl, dimethylpyridyl, methylquinolyl, dimethylpyrryl, methoxyfuryl, dimethoxypyridyl, difluoropyridyl, methylthiophenyl, isopropylthiophenyl or tert-butylthiophenyl.

The substituents listed specifically can in each case be interrupted at any desired position by one or more heteroatoms, where the number of these heteroatoms is not more than 10, preferably not more than 8, very particularly preferably not more than 5 and in particular not more than 3, and/or can in each case be substituted at any desired position, but not more than five, times, preferably not more than four times and particularly preferably not more than three times, by alkyl, alkyloxy, alkyloxycarbonyl, aryl, aryloxy, aryloxycarbonyl, hydroxycarbonyl, aminocarbonyl, heterocycles, heteroatoms or halogen atoms, where these can likewise be substituted a maximum of twice, preferably a maximum of once, by the specified groups.

The compound classes alkyl, aryl and heterocycles specified in this group have the meaning given above.

Heteroatoms are oxygen, nitrogen, sulfur or phosphorus.

Alkyloxy is a straight-chain or branched alkyl group having 1 to 20 carbon atoms (as specified above) which are bonded on via an oxygen atom (—O—), preferably C₁-C₁₀-alkyloxy, such as, for example, methoxy, ethoxy, propoxy.

Alkoxycarbonyl is an alkoxy group having 1 to 20 carbon atoms (as specified above), which is bonded on via a carbonyl group (—CO—), preferably C₁-C₁₀-alkyloxycarbonyl.

Aryloxy is a mono- to trinuclear aromatic ring system (as specified above) which is bonded on via an oxygen atom (—O—), preferably a mono- to binuclear, particularly preferably a mononuclear, aromatic ring system.

Aryloxycarbonyl is a mono- to trinuclear aryloxy group (as specified above), which is bonded on via a carbonyl group (—CO—), preferably a mono- to binuclear, particularly preferably a mononuclear, aryloxycarbonyl.

Halogen atoms are fluorine, chlorine, bromine and iodine.

In the case of the specified aliphatic substituents, the radicals R³ and R⁴ can also be joined to one another and thus together form a three- to eight-membered, preferably a five- to seven-membered and particularly preferably a five- to six-membered ring.

Furthermore, the substituents can in each case be interrupted at any desired position by one or more heteroatoms, where the number of these heteroatoms is not more than 10, preferably not more than 8, particularly preferably not more than 5 and in particular not more than 3. Heteroatoms are oxygen, nitrogen, sulfur or phosphorus.

In one preferred embodiment, the radicals R³, R⁴ and R⁵ are in each case independently of one another hydrogen or C₁-C₁₀-alkyl, preferably hydrogen or C₁-C₆-alkyl, and especially preferably hydrogen or C₁-C₄-alkyl. R³, R⁴ and R⁵ are very particularly preferably hydrogen, methyl or ethyl.

In a particularly preferred embodiment, the radicals R³, R⁴ and R⁵ are identical and are hydrogen, methyl or ethyl.

In a preferred embodiment, the radicals R¹ and R² are, in each case independently of one another, hydrogen or C₁-C₁₀-alkyl, preferably hydrogen or C₁-C₆-alkyl, and especially preferably hydrogen or C₁-C₄-alkyl. Very particularly preferably, R¹ and R² are, independently of one another, hydrogen or methyl.

Those units which are bracketed by the variables m and n can be present in any desired order.

If m≧2, those units which are bracketed by the variables m can in each case carry different or identical radicals R¹ and R², so that such N-hydroxyalkylated imidazoles (I) comprise ethylene oxide and propylene oxide units in any desired order.

In another preferred embodiment, m and n are in each case an integer from 0 to 10, particularly preferably 0 to 8 and especially preferably 0 to 4, where the above-mentioned M and n may not be 0 at the same time.

Suitable monomers are the (meth)acrylic acid esters of N-hydroxyalkylated monoalkylated imidazoles such as, for example, N-hydroxymethylimidazole and N-hydroxyethylimidazole. Further suitable monomers are the (meth)acrylic acid esters of imidazoles which comprise two or more alkoxy units, preferably exactly two alkoxy units.

Various ways of producing the above mentioned compounds (I), for example by esterification of compounds (II) with (meth)acrylic acid, are described in the European patent application No. 07102757.7, to which reference is hereby made in its entirety.

Preferred polymers comprise compounds of the formula (I) in copolymerized form, for which the following conditions apply.

| R³ | R⁴ | R⁵ | R⁶ | R⁷ | A | q | p |
|---|---|---|---|---|---|---|---|
| H | H | H | CH₃ | CH₂ | O | 1 | 1 |
| H | H | H | CH₃ | C₂H₄ | O | 1 | 1 |
| H | H | H | CH₃ | CH₂ | NH | 1 | 1 |
| H | H | H | CH₃ | C₂H₄ | NH | 1 | 1 |
| H | H | H | CH₃ | — | — | 0 | 0 |
| H | H | H | H | CH₂ | O | 1 | 1 |
| H | H | H | H | C₂H₄ | O | 1 | 1 |
| H | H | H | H | CH₂ | NH | 1 | 1 |
| H | H | H | H | C₂H₄ | NH | 1 | 1 |
| H | H | H | H | — | — | 0 | 0 |

Further preferred polymers comprise (meth)acrylic acid esters of N-hydroxyalkylated imidazoles in copolymerized form.

Particularly preferred polymers comprise (2-(1-imidazolyl)ethyl)methacrylate) in copolymerized form (R⁶═CH₃, R³ to R⁵═H, p=q1, A=O and R⁷═C₂H₄). The production of 2-(1-imidazolyl)ethyl methacrylate is known to the person skilled in the art and described, for example, in the European patent application No. 07102757.7.

The polymers can comprise, in copolymerized form, the compounds of the formula (I) also in the respective cationic form of the formula (Ia). Here, R⁸ is preferably CH₃ or C₂H₅. The compounds of the formula (Ia) can be produced by quaternization with alkylating agents. Suitable alkylating agents are, for example, C₅-C₃₀-alkyl halides, in particular bromides and chlorides, preference being given to C₁-C₄-alkyl halides or sulfates, such as ethyl chloride, ethyl bromide, methyl chloride, methyl bromide, dimethyl sulfate and diethyl sulfate. A preferred quaternizing agent is methyl chloride. A further preferred quaternizing agent is diethyl sulfate,

Charged cationic groups can also be produced by profanation of the amine nitrogen atoms with acids. Suitable acids are, for example, carboxylic acids, for example, lactic acid, or mineral acids, for example, phosphoric acid, sulfuric acid and hydrochloric acid. The cationic charge of compounds of the formula (Ia) is of course regularly neutralized by anions. These anions are preferably chloride, bromide, methylsulfate (CH₃—O—SO₃⁻ anion), ethylsulfate (C₂H₅—O—SO₃⁻ anion) and sulfate (—SO₄²⁻).

The polymers described above can also be converted to the cationic form following their production by partial or complete quaternization and/or protonation. In general, the same quaternizing agents are suitable for this purpose as for the quaternization of the monomeric compounds.

The copolymerized compounds of the formula (I) can be partially or completely quaternized in the polymer.

In a preferred embodiment of the invention, homopolymers of the compounds (I) and (Ia) are used in cosmetic preparations.

In one preferred embodiment of the invention, copolymers of the compounds (I) and (Ia) are used. These copolymers are obtainable through the polymerization of monomer mixtures which, besides the compounds of the formula (I) and (Ia), comprise further radically polymerizable compounds.

In principle, all radically polymerizable compounds are suitable for this purpose provided cosmetically acceptable polymers can be synthesized.

Preferred further radically polymerizable compounds are, for example, open-chain N-vinylamide compounds, such as N-vinylformamide, N-vinyl-N-methylformamide, N-vinylacetamide, M-vinyl-N-methylacetamide, N-vinyl-N-ethylacetamide, N-vinylpropionamide, N-vinyl-N-methylpropionamide, N-vinylbutyramide and mixtures thereof.

Further preferred radically polymerizable compounds are selected from the group consisting of acrylamide, the N-vinyl derivatives of optionally alkyl-substituted 2-pyrrolidone, optionally alkyl-substituted 2-piperidone and optionally alkyl-substituted ε-caprolactam.

Further preferred radically polymerizable compounds are selected from the group consisting of the N-vinyl derivatives of 2-pyrrolidone, 3-methyl-2-pyrrolidone, 4-methyl-2-pyrrolidone, 5-methyl-2-pyrrolidone, 3-ethyl-2-pyrrolidone, 3-propyl-2-pyrrolidone, 3-butyl-2-pyrrolidone, 3,3-dimethyl-2-pyrrolidone, 3,5-dimethyl-2-pyrrolidone, 5,5-dimethyl-2-pyrrolidone, 3,3,5-trimethyl-2-pyrrolidone, 5-methyl-5-ethyl-2-pyrrolidone, 3,4,5-trimethyl-2-pyrrolidone, 3-methyl-2-piperidone, 4-methyl-2-piperidone, 5-methyl-2-piperidone, 6-methyl-2-piperidone, 6-ethyl-2-piperidone, 3,5-dimethyl-2-piperidone, 4,4-dimethyl-2-piperidone, 3-methyl-ε-caprolactam, 4-methyl-ε-caprolactam, 5-methyl-ε-caprolactam, 6-methyl-ε-caprolactam, 7-methyl-ε-caprolactam, 3-ethyl-ε-caprolactam, 3-propyl-ε-caprolactam, 3-butyl-ε-caprolactam, 3,3-dimethyl-ε-caprolactam, 7,7-dimethyl-ε-caprolactam and mixtures thereof.

As at least one further radically polymerizable compound, particular preference is given to N-vinylpyrrolidone, methyl-N-vinylpyrrolidone or N-vinylcaprolactam.

Preferred further radically polymerizable compounds are esters of (meth)acrylic acid with amino alcohols substituted once or twice by C₁-C₂₄-alkyl on the nitrogen. These are particularly preferably selected from the group consisting of N-methylaminoethyl (meth)acrylate, N-ethylaminoethyl(meth)acrylate, N-(n-propyl)aminoethyl (meth)acrylate, N-(n-butyl)aminoethyl(meth)acrylate, N-(tert-butyl)aminoethyl (meth)acrylate, N,N-dimethylaminomethyl(meth)acrylate, N,N-dimethylaminoethyl (meth)acrylate, N,N-diethylaminoethyl(meth)acrylate, N,N-dimethylaminopropyl (meth)acrylate, N,N-diethylaminopropyl(meth)acrylate and N,N-dimethylamino-cyclohexyl(meth)acrylate.

Particular preference is given to N,N-dimethylaminoethyl methacrylate,

Further preferred radically polymerizable compounds are amides of (meth)acrylic acid with diamines substituted once or twice by C₁-C₂₄-alkyl on the nitrogen. These are particularly preferably selected from the group consisting of N-[2-(dimethyl-amino)ethyl]acrylamide, N-[2-(dimethylamino)ethyl]methacrylamide, N-[3-(dimethyl-amino)propyl]acrylamide, N-[3-(dimethylamino)propyl]methacrylamide, N-[4-(dimethyl-amino)butyl]acrylamide, N-[4-(dimethylamino)butyl]methacrylamide, N-[2-(diethyl-amino)ethyl]acrylamide, N-[4-(dimethylamino)cyclohexyl]acrylamide, N-[4-(dimethyl-amino)cyclohexyl]methacrylamide, N-[8-(dimethylamino)octyl]methacrylamide, N-[12-(dimethylamino)dodecyl]methacrylamide, N-[3-(diethylamino)propyl]meth-acrylamide and N-[3-(diethylamino)propyl]acrylamide.

Particular preference is given to N-[3-(dimethylamino)propyl]methacrylamide.

Suitable radically polymerizable compounds are also N,N-diallylamines and N,N-diallyl-N-alkylamines and acid addition salts and quaternization products thereof. Alkyl is here preferably C₁-C₂₄-alkyl. Preference is given to N,N-diallyl-N-methylamine and N,N-diallyl-N,N-dimethylammonium compounds, such as, for example, the chlorides and bromides. These include in particular N,N-diallyl-N-methylamine and its methylated derivative N,N-diallyl-N,N-dimethylammonium chloride (DADMAC).

Suitable monomers are also vinyl- and allyl-substituted nitrogen heterocycles, such as 2- and 4-vinylpyridine, 2- and 4-allylpyridine, and the salts thereof.

Furthermore, radically polymerizable compounds which may be copolymerized are α,β-ethylenically unsaturated monomers of the general formula (III)

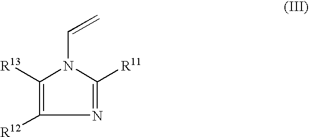

where R¹¹ to R¹³, independently of one another, are hydrogen, C₁-C₄-alkyl or phenyl.

Examples of suitable compounds of the general formula (III) can be found in Table 1 below:

TABLE 1

| R¹¹ | R¹² | R¹³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

Preference is given to 1-vinylimidazole (N-vinylimidazole) and mixtures which comprise N-vinylimidazole.

Suitable radically polymerizable compounds are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Suitable radically polymerizable compounds are also 2-hydroxyethylacrylamide, 2-hydroxyethylmethacrylamide, 2-hydroxyethylethacsylamide, 2-hydroxypropyl-acrylamide, 2-hydroxypropylmethacrylamide, 3-hydroxypropylacrylamide, 3-hydroxy-propylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

Suitable radically polymerizable compounds are also polyether acrylates, which, for the purposes of this invention, are generally understood as meaning esters of α,β-ethylenically unsaturated mono- and dicarboxylic acids with polyetherols. Suitable polyetherols are linear or branched substances having terminal hydroxyl groups and comprise ether bonds. In general, they have a molecular weight in the range from about 150 to 20 000. Suitable polyetherols are polyalkylene glycols, such as polyethylene glycols, polypropylene glycols, polytetrahydrofurans and alkylene oxide copolymers. Suitable alkylene oxides for producing alkylene oxide copolymers are, for example, ethylene oxide, propylene oxide, epichlorohydrin, 1,2- and 2,3-butylene oxide. The alkylene oxide copolymers can comprise the copolymerized alkylene oxide units in random distribution or in the form, of blocks. Preference is given to ethylene oxide/propylene oxide copolymers.

Suitable radically polymerizable compounds are also polyether acrylates of the general formula IV

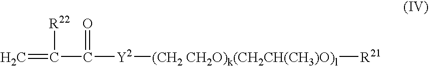

in which

the order of the alkylene oxide units is arbitrary,

k and l, independently of one another, are an integer from 0 to 1000, where the sum of k and l is at least 5,

R²¹ is hydrogen, C₁-C₃₀-alkyl, C₅-C₈-cycloalkyl or aryl,

R²² is hydrogen or C₁-C₈-alkyl,

Y² is O or NR²³, where R²³ is hydrogen, C₁-C₃₀-alkyl or C₅-C₅-cycloalkyl.

Preferably, k is an integer from 1 to 500, in particular 3 to 250. Preferably, l is an integer from 0 to 100. Preferably, R²² is hydrogen, methyl, ethyl, n-propyl, isopropyl, n-butyl, sec-butyl, tert-butyl, n-pentyl or n-hexyl, in particular hydrogen, methyl or ethyl. Preferably, R²¹ in the formula IV is hydrogen, methyl, ethyl, n-propyl, isopropyl, n-butyl, sec-butyl, n-pentyl, n-hexyl, octyl, 2-ethylhexyl, decyl, lauryl, palmityl or stearyl, phenyl. Preferably, Y² in the formula IV is O or NH.

Suitable polyether acrylates of the abovementioned type are, for example, the polycondensation products of the abovementioned α,β-ethylenically unsaturated mono- and/or dicarboxylic acids and their acid chlorides, amides and anhydrides with polyetherols. Suitable polyetherols can be readily produced by reacting ethylene oxide, 1,2-propylene oxide and/or epichlorohydrin with a starter molecule, such as water or a short-chain alcohol R²¹—OH. The alkylene oxides can be used individually, alternately one after the other or as a mixture. The polyether acrylates can be used on their own or in mixtures for producing the polymers used according to the invention.

Suitable additional radically polymerizable compounds are methyl(meth)acrylate, methyl ethacrylate, ethyl(meth)acrylate, ethyl ethacrylate, n-propyl(meth)acrylate, isopropyl(meth)acrylate, n-butyl(meth)acrylate, tert-butyl methacrylate, tert-butyl ethacrylate, n-octyl(meth)acrylate, 1,1,3,3-tetramethylbutyl(meth)acrylate, ethylhexyl (meth)acrylate, n-nonyl(meth)acrylate, n-decyl(meth)acrylate, n-undecyl (meth)acrylate, tridecyl(meth)acrylate, myristyl(meth)acrylate, pentadecyl (meth)acrylate, palmityl(meth)acrylate, heptadecyl(meth)acrylate, nonadecyl (meth)acrylate, arrachinyl(meth)acrylate, behenyl(meth)acrylate, lignocerenyl (meth)acrylate, cerotinyl(meth)acrylate, melissinyl(meth)acrylate, palmitoleinyl (meth)acrylate, oleyl(meth)acrylate, linolyl(meth)acrylate, linolenyl(meth)acrylate, stearyl(meth)acrylate, lauryl(meth)acrylate and mixtures thereof.

Preferred radically polymerizable compounds are the esters of α,β-ethylenically unsaturated mono- and dicarboxylic acids with C₁-C₄-alkanols.

Suitable additional radically copolymerizable compounds are also vinyl acetate, vinyl propionate, vinyl butyrate and mixtures thereof.

Suitable additional radically copolymerizable compounds are also methyl vinyl ether, ethyl vinyl ether, n-propyl vinyl ether, isopropyl vinyl ether, n-butyl vinyl ether, isobutyl vinyl ether, 2-ethylhexyl vinyl ether, dodecyl vinyl ether and octadecyl vinyl ether.

Suitable additional radically copolymerizable compounds are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

Preferred radically copolymerizable compounds are compounds with a radically polymerizable, α,β-ethylenically unsaturated double bond and at least one anionogenic and/or anionic group per molecule.

Preferred radically copolymerizable compounds are compounds which are selected from monoethylenically unsaturated carboxylic acids, sulfonic acids, phosphonic acids and mixtures thereof. These include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, methacrylic anhydride, ethacrylic acid, α-chloroacrylic acid, crotonic acid, maleic acid, maleic anhydride, itaconic acid, citraconic acid, mesaconic acid, glutaconic acid, aconitic acid and fumaric acid. The compounds with a radically polymerizable, α,β-ethylenically unsaturated double bond and at least one anionogenic and/or anionic group per molecule further include the half-esters of monoethylenically unsaturated dicarboxylic acids having 4 to 10, preferably 4 to 6, carbon atoms, e.g. of maleic acid, such as monomethyl maleate. The compounds with a radically polymerizable, α,β-ethylenically unsaturated double bond and at least one anionogenic and/or anionic group per molecule also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. Furthermore, these also include the salts of the abovementioned acids, in particular the sodium, potassium and ammonium salts, and also the salts with amines. These monomers can be used as such or as mixtures. If such an anionic or anionogenic compound is selected for the copolymerization, then it is preferably selected from the group consisting of acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid, 2-acrylamido-2-methylpropanesulfonic acid, vinylphosphonic acid and mixtures thereof. The anionic or anionogenic compound used for the copolymerization is particularly preferably selected from acrylic acid, methacrylic acid and mixtures thereof.

A preferred embodiment of the invention is the use according to the invention of the polymers described above which comprise at least one anionic or anionogenic compound in copolymerized form.

A further preferred embodiment of the invention is the use according to the invention of the polymers described above which comprise at least one cationic or cationogenic compound in copolymerized form.

A further preferred embodiment of the invention is the use according to the invention of the polymers described above which comprise at least one nonionic compound in copolymerized form.

A further preferred embodiment of the invention is the use according to the invention of the polymers described above which comprise at least one anionic or anionogenic compound and at least one cationic or cationogenic compound in copolymerized form.

A further preferred embodiment of the invention is the use according to the invention of the polymers described above which comprise at least one anionic or anionogenic compound and at least one nonionic compound in copolymerized form.

A further preferred embodiment of the invention is the use according to the invention of the polymers described above which comprise at least one cationic or cationogenic compound and at least one nonionic compound in copolymerized form.

A further preferred embodiment of the invention is the use according to the invention of the polymers described above which comprise at least one anionic or anionogenic compound and at least one cationic or cationogenic compound and at least one nonionic compound in copolymerized form.

Preference is given to the use according to the invention of the polymers where the at least one further monomer is selected from N-vinyllactams, monomers comprising acid groups, (meth)acrylic acid esters, (meth)acrylamides, vinyl ethers and diallylamines.

Particular preference is given to the use according to the invention of the polymers where the at least one further monomer is selected from N-vinylpyrrolidone, acrylic acid, methyl methacrylate, N,N-diallyl-N,N-dimethylammonium chloride, N,N-dimethyl-aminoethyl methacrylate, N-[3-(dimethylamino)propyl]methacrylamide, methacrylamide and mixtures thereof.

Preference is given to the use according to the invention, where the polymers comprise at least two further monomers in copolymerized form, where at least one of the further monomers is selected from N-vinyllactams and at least one of the further monomers is selected from (meth)acrylamides.

Particular preference is given to the use according to the invention of polymers analogous to Table 2, page 43 of WO 03/092640, where according to the invention the vinylimidazole copolymerized therein is replaced partially or completely by ImEMA, Reference is hereby made to the disclosure of WO 03/092640 in its entirety.

Crosslinkers

The copolymers used according to the invention can, if desired, comprise, in copolymerized form, at least one crosslinker, i.e. a compound with two or more than two ethylenically unsaturated, nonconjugated double bonds.

Preferably, crosslinkers are used in an amount of from 0.01 to 2% by weight, particularly preferably 0.1 to 1% by weight, based on the total weight of the monomers used for the polymerization.

Suitable crosslinkers are, for example, acrylic esters, methacrylic esters, allyl ethers or vinyl ethers of at least dihydric alcohols. The OH groups of the parent alcohols here may be completely or partially etherified or esterified; however, the crosslinkers comprise at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalinic acid neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and also polyethylene glycols, polypropylene glycols and polytetrahydrofurans having molecular weights of in each case 200 to 10 000. Apart from the homopolymers of ethylene oxide or propylene oxide, it is also possible to use block copolymers of ethylene oxide or propylene oxide or copolymers which comprise ethylene oxide and propylene oxide groups in incorporated form. Examples of parent alcohols having more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates, respectively. The polyhydric alcohols can also firstly be converted to the corresponding glycidyl ethers by reaction with epichlorohydrin.

Further suitable crosslinkers are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the monohydric, unsaturated alcohols with polybasic carboxylic acids, for example malonic acid, tartaric acid, trimellitic add, phthalic acid, terephthalic acid, citric acid or succinic acid.

Further suitable crosslinkers are esters of unsaturated carboxylic acids with the above-described polyhydric alcohols, for example oleic acid, crotonic acid, cinnamic acid or 10-undecenoic acid.

Further suitable crosslinkers are urethane diacrylates and urethane polyacrylates, as are commercially available, for example, under the name Laromer®.

Furthermore suitable as crosslinker are straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Also suitable as crosslinkers are the acrylamides, methacrylamides and N-allylamines, of at least difunctional amines. Such amines are, for example, 1,2-diaminomethane, 1,2-diaminoethane, 1,3-diaminopropane, 1,4-diaminobutane, 1,8-diaminohexane, 1,12-dodecanediamine, piperazine, diethylenetriamine or isophoronediamine. Likewise suitable are the amides of allylamines and unsaturated carboxylic acids, such as acrylic acid, methacrylic acid, itaconic acid, maleic acid, or at least dibasic carboxylic acids, as have been described above.

Also suitable as crosslinkers are triallylamine and triallylmonoalkylammonium salts, e.g. triallylmethylammonium chloride or methylsulfate.

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Further suitable crosslinkers are divinyldioxane, tetraallylsilane or tetravinylsilane.

It is of course also possible to use mixtures of the abovementioned compounds. Particularly preferably used crosslinkers are, for example, methylenebisacrylamide, triallylamine and triallylalkylammonium salts, divinylimidazole, pentaerythritol triallyl ether, N,N′-divinylethyleneurea, trimethylolpropane diallyl ether, allylsucrose, reaction products of polyhydric alcohols with acrylic acid or methacrylic acid, methacrylic acid esters and acrylic acid esters of polyalkylene oxides or polyhydric alcohols which have been reacted with ethylene oxide and/or propylene oxide and/or epichlorohydrin.

Very particularly preferred crosslinkers are pentaerythritol triallyl ether, trimethylolpropane diallyl ether, allylsucrose, methylenebisacrylamide, N,N′-divinylethyleneurea, triallylamine and triallylmonoalkylammonium salts and acrylic acid esters of glycol, butanediol, trimethylolpropane or glycerol or acrylic acid esters of glycol, butanediol, trimethylolpropane or glycerol reacted with ethylene oxide and/or epichlorohydrin.

In the case of the presence of quaternizable groups in the polymer, the crosslinking can also take place by reacting the polymer with alkylating agents which are suitable for two-fold alkylation.

The uncrosslinked polymers suitable for the use according to the invention preferably have mass-average molecular weights M_{w} in the range from 10 000 to 5 million, further preferably from 30 000 to 2 million, particularly preferably from 500 000 to 1.5 million g/mol.

The uncrosslinked polymers suitable for the use according to the invention preferably have number-average molecular weights Mₙ in the range from 10 000 to 500 000, further preferably from 40 000 to 250 000 g/mol.

Solution Polymerization

Preferably, the polymers are prepared by solution polymerization in aqueous solution. In a further embodiment of the invention, the polymers are produced by solution polymerization in alcoholic solution.

In a further embodiment of the invention, the solvent comprises water and alcohol. Suitable alcohols are, for example, methanol, ethanol, n-propanol, isopropanol, n-butanol, sec-butanol, tert-butanol, 3-methyl-1-butanol (isoamyl alcohol), n-hexanol, cyclohexanol or glycols, such as ethylene glycol, propylene glycol and butylene glycol, and alkyl ethers of polyhydric alcohols, such as diethylene glycol, methylene glycol, polyethylene glycols with number-average molecular weights up to about 3000, glycerol.

Particularly preferably, the alcohol is or comprises ethanol and/or isopropanol, in particular isopropanol. The alcohol fraction of the total amount of solvent is in the range from 0 to 80% by weight, preferably from 10 to 70% by weight, particularly preferably from 15 to 50% by weight, in each case based on the total amount of solvent A further customary solvent comprises in the region of 60-80% by weight of alcohol and about 40-20% by weight of water.

In addition to water or water/alcohol, further solvents may be present in the polymerization solution. Of suitability in principle are all solvents suitable for the radical polymerization, such as, for example, acetone, acetonitrile, aniline, anisole, benzonitrile, tert-butyl methyl ether (TBME), gamma-butyrolactone, quinoline, chloroform, cyclohexane, diethyl ether, dimethylacetamide, dimethylformamide, dimethyl sulfoxide, dioxane, ethyl acetate, ethylene dichloride, ethylene glycol dimethyl ether, formamide, hexane, methylene chloride, methyl ethyl ketone, N-methyl-formamide, petroleum ether/light benzine, propylene carbonate (4-methyl-1,3-dioxol-2-one), sulfolane, tetrachloroethene, tetrachloromethane, tetrahydrofuran, toluene, 1,1,1-trichloroethane, trichloroethene, methylene glycol dimethyl ether (triglyme) and mixtures thereof.

Polymerization solution is understood as meaning the substance mixture which is present following the addition of all of the components and following completion of the polymerization and before the first work-up step, such as, for example, a drying, a neutralization or a steam distillation.

The amount of water is preferably in the range from 100 to 50% by weight based on the solvent.

The amount of alcohol is preferably in the range from 0 to 50% by weight based on the solvent.

The amount of substances furthermore present in the polymerization solution, which are essentially the monomers, the initiator and, if appropriate, regulator and crosslinker, is preferably at least 5% by weight, particularly preferably at least 10% by weight and in particular at least 20% by weight and preferably at most 55% by weight, particularly preferably at most 50% by weight and in particular at most 45% by weight, of the polymerization solution.

This amount is also referred to as solids content of the polymerization solution.

Preference is given to a method according to the invention in which the temperature of the polymerization solution is in the range from 30° C. to 120° C., particularly preferably in the range from 50° C. to 110° C. and in particular in the range from 60° C. to 90° C.

The polymerization usually takes place under atmospheric pressure, although it can also proceed under reduced or increased pressure. A suitable pressure range is between 1 and 10 bar.

Precipitation Polymerization

The polymers to be used according to the invention can also be produced by radical precipitation polymerization. The method of precipitation polymerization is known per se to the person skilled in the art. In precipitation polymerization, the monomers used are soluble in the reaction medium (monomer, solvent), but the corresponding polymer is not. The resulting polymer is insoluble under the chosen polymerization conditions and precipitates out of the reaction mixture. The precipitation polymerization preferably takes place in an ester, such as ethyl acetate or butyl acetate, as solvent. The resulting polymer particles precipitate out of the reaction solution and can be isolated by customary methods, such as filtration using subatmospheric pressure. For the precipitation polymerization it is possible to use surface-active, polymeric compounds, preferably based on polysiloxane. In the case of precipitation polymerization, polymers having higher molecular weights are generally obtained than in the case of solution polymerization.

The initiator used for the radical polymerization in solution in water is preferably at least one water-soluble polymerization initiator selected from the group consisting of peroxides, hydroperoxides, peroxodisulfates, percarbonates, peroxide esters, azo compounds and mixtures thereof.

A water-soluble polymerization initiator is understood as meaning an initiator which is soluble at 20° C. and 1013 mbar to at least 1 g, preferably to at least 10 g, in 1 liter of water.

In a preferred embodiment of the invention, the water-soluble polymerization initiator is selected from the group consisting of water-soluble azo compounds, hydrogen peroxide, lithium peroxodisulfate, sodium peroxodisulfate, potassium peroxodisulfate, ammonium peroxodisulfate and mixtures thereof.

The water-soluble polymerization initiator is further preferably selected from the group consisting of

2,2′-azobis(2-methylpropionamidine)dihydrochloride

2,2′-azobis[2-(5-methyl-2-imidazolin-2-yl)propane]dihydrochloride

2,2′-azobis[2-(2-imidazolin-2-yl)propane]dihydrochloride

2,2′-azobis[2-(2-imidazolin-2-yl)propane disulfate dihydrate

2,2-azobis(2-methylpropionamide)dihydrochloride

2,2′-azobis[N-(2-carboxyethyl)-2-methylpropionamidine]tetrahydrate

2,2′-azobis[2-(3,4,5,6-tetrahydropyrimidin-2-yl)propane]dihydrochloride

2,2′-azobis{2-[1-(2-hydroxyethyl)-2-imidazolin-2-yl]propane}dihydrochloride

2,2′-azobis[2-(2-imidazolin-2-yl)propane]

2,2′-azobis{2-methyl-N-[1,1-bis(hydroxymethyl)-2-hydroxyethyl]propionamide

2,2′-azobis{2-methyl-N-[2-(1-hydroxybutyl)]propionamide}

2,2′-azobis[2-methyl-N-(2-hydroxyethyl)propionamide] and mixtures thereof.

Water-soluble redox initiator systems can also be used as polymerization initiators. Such redox initiator systems comprise at least one peroxide-containing compound in combination with a redox coinitiator, for example reductive sulfur compounds, for example bisulfites, sulfites, thiosulfates, dithionites and tetrathionates of alkali metals and ammonium compounds. Thus, combinations of peroxodisulfates with alkali metal or ammonium hydrogen sulfites can be used, e.g. ammonium peroxodisulfates and ammonium disulfite. The amount of the peroxide-containing compound relative to the redox coinitiator is in the range from 301 to 0.05:1.

In combination with the initiators or the redox initiator systems, transition metal catalysts can additionally be used, e.g. salts of iron, cobalt, nickel, copper, vanadium and manganese. Suitable salts are, for example, iron(II) sulfate, cobalt(II) chloride, nickel(II) sulfate, or copper(I) chloride. Based on the monomers, the reductive transition metal salt is used in a concentration of from 0.1 ppm to 1000 ppm. It is thus possible to use combinations of hydrogen peroxide with iron(II) salts, such as, for example, 0.5 to 30% hydrogen peroxide and 0.1 to 500 ppm of Mohr's salt.

Furthermore, redox coinitiators and/or transition metal catalysts can be co-used in combination with the abovementioned initiators, e.g. benzoin, dimethylaniline, ascorbic acid, and complexes of heavy metals, such as copper, cobalt, iron, manganese, nickel and chromium. The amounts of redox coinitiators or transition metal catalysts usually used are about 0.1 to 1000 ppm, based on the amounts of monomers used. Further suitable initiators are described in chapters 20 and 21 of Macromolecules, Vol. 2, 2nd Ed., H. G. Elias, Plenum Press, 1984, New York, to which reference is made here in its entirety. Furthermore, suitable photoinitiators are described in S. P. Pappas, J. Rad. Cur., July 1987, p. 8, to which reference is made here in its entirety.

Further initiators suitable for producing the polymers to be used according to the invention are selected from the group consisting of diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleinate, cumene hydroperoxide, diisopropyl peroxydicarbamate, bis(o-toluoyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, or 2-2′-azobis(2-methylbutyronitrile), diisobutyryl peroxide, cumyl peroxyneodecanoate, cumyl peroxyneoheptanoate, tert-amyl peroxyneodecanoate, tert-butyl peroxyneodecanoate, di-sec-butyl peroxydicarbonate, diisopropyl peroxydicarbonate, diacetyl peroxydicarbonate, tert-amyl peroxyperpivalate, 2,5-dimethyl-2,5-di(tert-butylperoxy)hexane, tert-butyl peroxyacetate, tert-butyl peroxybenzoate.

The amount of the at least one water-soluble initiator used for the polymerization of the monomers is preferably from 0.0001 to 10% by weight, particularly preferably 0.001 to 5% by weight and in particular 0.02 to 3% by weight, based on the total amount of the monomers used.

To adjust the molecular weight, the polymerization can take place in the presence of at least one regulator. Regulators which can be used are the customary compounds known to the person skilled in the art, such as, for example, sulfur compounds, e.g. mercaptoethanol, cystein, 2-ethylhexyl thioglycolate, thioglycolic acid or dodecyl mercaptan, and also tribromochloromethane or other compounds which have a regulating effect on the molecular weight of the resulting polymers.

The solution polymerization can be carried out either as a batch process or in the form of a feed method, including monomer feed, stepwise procedure and gradient procedure. Preference is generally given to the feed method in which, if appropriate, some of the polymerization mixture is initially introduced and heated to the polymerization temperature and then the remainder of the polymerization mixture is introduced usually via one or more spatially separate feeds, continuously, stepwise or with overlap of a concentration gradient while maintaining the polymerization of the polymerization zone.

In one embodiment of the invention, the total amount of the monomers and, if appropriate, of the crosslinkers and of the regulators is initially introduced and the initiator is gradually added to the reaction mixture.

In a further embodiment of the invention, some of a monomer, for example 1 to 50% by weight, preferably 5 to 40% by weight, particularly preferably 10 to 30% by weight, of a monomer, solvent and some of the regulator used if appropriate, for example 1 to 50% by weight, preferably 5 to 40% by weight, particularly preferably 10 to 30% by weight, of the regulator is initially introduced, and the other monomers, solvent and the remaining regulator in one feed, and solvent and initiator in a second feed are gradually added to the reaction mixture. In this connection, it is advantageous to use solvents both in the initial charge and also in the feeds.

In a further embodiment of the invention, the majority of a monomer, for example more than 50% by weight, preferably more than 80% by weight, further preferably more than 70% by weight, particularly preferably more than 80% by weight and in particular more than 90% by weight, is initially introduced and, if appropriate, heated together with solvent. Then, some of the monomers, for example up to 50% by weight, preferably up to 40% by weight, particularly preferably up to 30% by weight and in particular up to 20% by weight, of the other monomers are added to the initial charge, heated if appropriate, and the polymerization is started with the help of the initiator. The remainder of the monomers and of the initiator is then gradually metered in. In a preferred embodiment, the other monomers are metered in together as a mixture.

The polymerization is preferably carried out largely with exclusion of oxygen. It is preferred to carry out the polymerization under protective gas atmosphere, such as, for example, argon atmosphere or preferably nitrogen atmosphere.

The polymerization can take place in principle at the pH resulting through the monomers used.

Preferably, the pH of the polymerization solution is adjusted to a value of from 5 to 10, further preferably 6 to 12, particularly preferably 6.5 to 7.5.

Preferably, the pH of the initial charge and of the various feeds is adjusted to a value of from 5 to 10, further preferably 6 to 12, particularly preferably 8.5 to 7.5.

It is furthermore advantageous to then keep the pH in this range during the polymerization. Of suitability for adjusting the pH before, during or after the polymerization are, in principle, all inorganic or organic bases and acids, in particular those which, apart from possible salt formation, do not react with the monomers. Suitable bases are, for example, alkali metal and alkaline earth metal hydroxides, ammonia, tertiary amines, such as triethylamine, and also amino alcohols, such as triethanolamine, methyldiethanolamine or dimethylethanolamine. For adjusting the pH, preference is given to using NaOH or at least one tertiary amine, which is selected in particular from N,N-dimethylethanolamine, N-methyldiethanolamine, triethanolamine and mixtures thereof. Suitable acids are organic acids, such as formic acid, acetic acid, butyric acid, lactic acid, and inorganic acids, such as, for example, phosphoric acid, hydrochloric acid, sulfuric acid or nitric acid, in any case, it is advantageous to keep the hydrolysis of the monomers as low as possible by establishing a pH in the range from 8 to 8.

To achieve the purest possible polymers with a low residual monomer content, the polymerization (main polymerization) can be followed by an after polymerization step. The after polymerization can take place in the presence of the same initiator system as the main polymerization, or a different initiator system. Preferably, the after polymerization takes place at least at the same temperature as the main polymerization, preferably at a higher temperature than the main polymerization. If desired, the reaction mixture can, after the polymerization or between the first and second polymerization steps, be subjected to stripping with steam or to steam distillation, which is particularly advantageously carried out to eliminate components with an undesired odor.

The monomers used for the polymerization are preferably converted to at least 95%, particularly preferably to at least 99% and in particular to at least 99.9% (degree of polymerization).

The polymers suitable for the use according to the invention can, however, also be produced by other customary polymerization methods, such as, for example, emulsion polymerization, precipitation polymerization, water-in-water emulsion polymerization, bulk polymerization. However, this list is neither exhaustive nor complete and should in no way limit the invention.

The polymers present in solution or dispersed form after the polymerization can be converted to powders by customary drying methods known to the person skilled in the art. Preferred methods are spray drying, spray fluidized-bed drying, drum drying and belt drying, it is likewise possible to use freeze-drying and freeze-concentration. If desired, solvents can also be partially or completely removed by customary methods, e.g. distillation at reduced pressure.

According to the invention, preference is given to using the polymers described above as conditioners, in particular in haircare preparations, or as hair-setting agents, in particular in haircare preparations, and also as thickeners.

The polymers to be used according to the invention can of course also be used in other technical fields. Examples of such further applications that may be mentioned are oil recovery, paper processing, detergents, paint, or coating. The invention further provides cosmetic or pharmaceutical compositions comprising

A) at least one polymer to be used according to the invention and

B) at least one cosmetically acceptable carrier.

The compositions according to the invention preferably have a cosmetically or pharmaceutical acceptable carrier B) which is chosen from

i) water,

ii) water-miscible organic solvents, preferably C₂-C₄-alkanols, in particular ethanol,

iii) oils, fats, waxes,

iv) esters of C₆-G₃₀-monocarboxylic acids with mono-, di- or trihydric alcohols that are different from iii),

v) saturated acyclic and cyclic hydrocarbons,

vi) fatty acids,

vii) fatty alcohols,

viii) propellant gases,

and mixtures thereof.

The compositions according to the invention have, for example, an oil or fatty component B) which is selected from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil or fat components B) are selected from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, pig grease, spermaceti, spermaceti oil, sperm oil, wheat germ oil, macadamia nut oil, evening primrose oil, jojoba oil: fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candelilla wax, spermaceti, and mixtures of the abovementioned oil or fat components.

Suitable cosmetically and pharmaceutical compatible oil and fat components B) are described in Karl-Heinz Schrader, Grundlagen und Rezepturen tier Kosmetika [Fundamentals and formulations of cosmetics], 2^{nd} edition, Verlag Hüthig, Heidelberg, pp. 319-355, to which reference is made here.

Advantageously, those oils, fats and/or waxes are selected which are described on page 28, line 39 to page 34, line 22 of WO 2006/106140. Reference is hereby made to the content of the specified passage in its entirety.

The content of further oils, fats and waxes is at most 50% by weight, preferably 30% by weight, further preferably at most 20% by weight, based on the total weight of the composition.

Suitable hydrophilic carriers B) are selected from water, mono-, di- or polyhydric alcohols having preferably 1 to 8 carbon atoms, such as ethanol, n-propanol, isopropanol, propylene glycol, glycerol, sorbitol, etc.

The cosmetic compositions according to the invention may be skin cosmetic, hair cosmetic, dermatological, hygiene or pharmaceutical compositions. On account of their film-forming properties, the above-described copolymers and polyelectrolyte complexes are suitable in particular as additives for hair and skin cosmetics.

Preferably, the compositions according to the invention are in the form of a gel, foam, spray, ointment, cream, emulsion, suspension, lotion, milk or paste. If desired, liposomes or microspheres can also be used.

The cosmetically or pharmaceutically active compositions according to the invention can additionally comprise cosmetically and/or dermatologically active ingredients, as well as auxiliaries.

Preferably, the cosmetic compositions according to the invention comprise at least one polymer to be used according to the invention as defined above, at least one carrier B) as defined above and at least one constituent different therefrom, which is selected from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolyzates, lipids, antioxidants, antifoams, antistats, emollients and softeners.

The cosmetic compositions according to the invention can be present as aqueous or aqueous-alcoholic solutions, O/W and W/O emulsions, hydrodispersion formulations, solids-stabilized formulations, stick formulations, PIT formulations, in the form of creams, foams, sprays (pump spray or aerosol), gels, gel sprays, lotions, oils, oil gels or mousse and accordingly can be formulated with customary further auxiliaries.

Particularly preferred cosmetic compositions for the purposes of the present invention are shampoos and haircare compositions. The invention accordingly also relates to compositions for the cleansing and/or care of the hair.

In particular, the invention relates to haircare compositions selected from the group consisting of pretreatment compositions, hair rinses, hair conditioners, hair balms, leave-on hair treatments, rinse-off hair treatments, hair tonics, pomades, styling creams, styling lotions, styling gels, end fluids, hot-oil treatments and foam treatments.

Furthermore, the invention relates to cosmetic compositions which are selected from gel creams, hydroformulations, stick formulations, cosmetic oils and oil gels, mascara, self-tanning compositions, facecare compositions, bodycare compositions, aftersun preparations, hair-shaping compositions and hair-setting compositions.

Further cosmetic compositions according to the invention are skin cosmetic compositions, in particular those for skincare. These are present in particular as W/O or O/W skin creams, day and night creams, eye creams, face creams, antiwrinkle creams, mimic creams, moisturizing creams, bleach creams, vitamin creams, skin lotions, care lotions and moisturizing lotions.

Furthermore, the polymer combinations according to the invention are suitable as ingredients for skin cosmetic preparations such as face tonics, face masks, deodorants and other cosmetic lotions and for use in decorative cosmetics, for example as concealing stick, stage make-up, in mascara and eye shadows, lipsticks, kohl pencils, eyeliners, make-up, foundations, blushers and powders and eyebrow pencils.

Furthermore, the compositions according to the invention can be used in nose strips for pore cleansing, in antiacne compositions, repellents, shaving compositions, hair removal compositions, personal hygiene compositions, footcare compositions, and also in babycare.

Further preferred compositions according to the invention are washing, showering and bathing preparations which comprise the polymers to be used according to the invention.

For the purposes of this invention, washing, showering and bathing preparations are understood as meaning soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and gels, foam baths, oil baths and scrub preparations, shaving foams, lotions and creams.

Suitable further ingredients for these washing, showering and bathing preparations according to the invention are described below.

Besides the polymers to be used according to the invention, the compositions comprise further cosmetically acceptable additives, such as, for example, emulsifiers and coemulsifiers, solvents, surfactants, oil bodies, preservatives, perfume oils, cosmetic care and active ingredients, such as AHA acids, fruit acids, ceramides, phytantriol, collagen, vitamins and provitamins, for example vitamin A, E and C, retinol, bisabolol, panthenol, natural and synthetic photoprotective agents, natural substances, opacifiers, solubility promoters, repellents, bleaches, colorants, tinting agents, tanning agents (e.g. dihydroxyacetone), micropigments, such as titanium oxide or zinc oxide, superfatting agents, pearlescent waxes, consistency regulators, thickeners, solubilizers, complexing agents, fats, waxes, silicone compounds, hydrotropes, dyes, stabilizers, pH regulators, reflectors, proteins and protein hydrolyzates (e.g. wheat, almond or pea proteins), caramide, protein hydrolyzates, salts, gel formers, consistency regulators, silicones, humectants (e.g. 1,2-pentanediol), retailing agents, UV photoprotective filters and further customary additives. Furthermore, to establish the properties desired in each case, it is in particular also possible for further polymers to be present.

The cosmetic compositions according to the invention comprise the polymers to be used according to the invention in an amount of from 0.01 to 10% by weight, preferably 0.05 to 5% by weight, particularly preferably 0.1 to 1.5% by weight, based on the weight of the composition.

In a preferred embodiment of the invention, the inventive washing, showering and bathing preparations, and shampoos and haircare compositions furthermore comprise at least one surfactant.

In a further preferred embodiment of the invention, besides the polymers to be used according to the invention, the shampoos and haircare compositions according to the invention furthermore comprise at least one oil and/or fatty phase and a surfactant.

Surfactants

Surfactants which can be used are anionic, cationic, nonionic and/or amphoteric surfactants.

Advantageous washing-active anionic surfactants for the purposes of the present invention are
acylamino acids and salts thereof, such as acyl glutamates, in particular sodium acyl glutamate
sarcosinates, for example myristoyl sarcosine, TEA lauroyl sarcosinate, sodium lauroyl sarcosinate and sodium cocoyl sarcosinate, sulfonic acids and salts thereof, such as
acyl isethionates, for example sodium or ammonium cocoyl isethionate
sulfosuccinates, for example dioctyl sodium sulfosuccinate, disodium laureth sulfosuccinate, disodium lauryl sulfosuccinate and disodium undecylenamido MEA sulfosuccinate, disodium PEG-5 lauryl citrate sulfosuccinate and derivatives,
alkyl ether sulfates, for example sodium, ammonium, magnesium, MIPA, TIPA laureth sulfates, sodium myreth sulfate and sodium C₁₂₋₁₃ pareth sulfate,
alkyl ether sulfonates, for example sodium C₁₂₋₁₅ pareth-15 sulfonate
alkyl sulfates, for example sodium, ammonium and TEA lauryl sulfate.

Further advantageous anionic surfactants are
taurates, for example sodium lauroyl taurate and sodium methyl cocoyl taurate,
ether carboxylic acids, for example sodium laureth-13 carboxylate and sodium PEG-6cocamide carboxylate, sodium PEG-7 olive oil carboxylate
phosphoric acid esters and salts, such as, for example, DEA oleth-10 phosphate and dilaureth-4 phosphate,
alkyl sulfonates, for example, sodium coconut monoglyceride sulfate, sodium C₁₂₋₁₄ olefinsulfonate, sodium lauryl sulfoacetate and magnesium PEG-3 cocamide sulfate,
acyl glutamates, such as di-TEA palmitoyl aspartate and sodium caprylic/capric glutamate,
acyl peptides, for example palmitoyl hydrolyzed milk protein, sodium cocoyl hydrolyzed soya protein and sodium/potassium cocoyl hydrolyzed collagen,
   and also carboxylic acids and derivatives, such as, for example, lauric acid, aluminum stearate, magnesium alkanolate and zinc undecylenate, ester carboxylic acids, for example, calcium stearoyl lactylate, laureth-6 citrate and sodium PEG-4 lauramide carboxylate
alkylarylsulfonates.

Advantageous washing-active cationic surfactants for the purposes of the present invention are quaternary surfactants. Quaternary surfactants comprise at least one N atom which is covalently bonded to 4 alkyl or aryl groups. For example, alkylbetaine, alkylamidopropylbetaine and alkylamidopropylhydroxysultaine are advantageous. Further advantageous cationic surfactants for the purposes of the present invention are also
alkylamines,
alkylimidazoles and
ethoxylated amines
   and in particular salts thereof.

Advantageous washing-active amphoteric surfactants for the purposes of the present invention are acyl/dialkylethylenediamines, for example sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut fatty acid amidoethyl N-hydroxyethylglycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous washing-active nonionic surfactants for the purposes of the present invention are
alkanolamides, such as cocamides MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers, alkyl polyglycosides, such as lauryl glucoside, decyl glycoside and cocoglyeoside, glycosides with an HLB value of at least 20 (e.g. Beisil® SPG 128V (Wacker)).

Further advantageous nonionic surfactants are alcohols and amine oxides, such as cocoamidopropylamine oxide.

Preferred anionic, amphoteric and nonionic shampoo surfactants are specified, for example, in “Kosmetik und Hygiene von Kopf bis Fuβ” [Cosmetics and Hygiene from Head to Toe], Ed. W. Umbach, 3^{rd} edition, Wiley-VCH, 2004, pp. 131-134, to which reference is made at this point in its entirety.

Among the alkyl ether sulfates, sodium alkyl ether sulfates based on di- or triethoxylated lauryl and myristyl alcohol in particular are preferred. They are clearly superior to the alkyl sulfates with regard to insensitivity toward water hardness, ability to be thickened, low-temperature solubility and, in particular, skin and mucosa compatibility. They can also be used as sole washing raw materials for shampoos. Lauryl ether sulfate has better foam properties than myristyl ether sulfate, but is inferior to this as regards mildness.

Alkyl ether carboxylates are often used in combination with alkyl ether sulfates and amphoteric surfactants in hair washing compositions.

Sulfosuccinic acid esters (sulfosuccinates) are mild and readily foaming surfactants, but, on account of their poor ability to be thickened, are preferably only used together with other anionic and amphoteric surfactants and, on account of their low hydrolysis stability, are preferably only used in neutral and well buffered products,

Amidopropylbetaines as sole washing raw materials are unimportant in practice since their foaming behavior and also their ability to be thickened are only moderate. On the other hand, these surfactants have excellent skin and eye mucosa compatibility. In combination with anionic surfactants, their mildness can be synergistically improved. Preference is given to the use of cocamidopropylbetaine.

Amphoacetates/amphodiacetates have, as amphoteric surfactants, very good skin and mucosa compatibility and can have a hair-conditioning effect and/or enhance the care effect of additives. Similarly to the betaines, they are used for the optimization of alkyl ether sulfate formulations. Sodium cocoamphoacetate and disodium cocoamphodiacetate are most preferred.

Alkyl polyglycosides are nonionic washing raw materials. They are mild, have good universal properties, but are weakly foaming. For this reason, they are preferably used in combinations with anionic surfactants.

Sorbitan esters are likewise types of nonionic washing raw materials. On account of their excellent mildness, they are preferably employed for use in baby shampoos. Being weak reamers, they are preferably used in combination with anionic surfactants. It is advantageous to select the washing-active surfactant or surfactants from the group of surfactants which have an HLB value of more than 25, those which have an HLB value of more than 35 being particularly advantageous.

According to the invention, it is advantageous if one or more of these surfactants is used in a concentration of from 1 to 30% by weight, preferably in a concentration of from 5 to 25% by weight and very particularly preferably in a concentration of from 10 to 20% by weight, in each case based on the total weight of the composition.

Polysorbates

As washing-active agents, polysorbates can also advantageously be incorporated into the compositions according to the invention.

Polysorbates advantageous for the purposes of the invention are, for example,
polyoxyethylene(20) sorbitan monolaurate (Tween® 20, CAS No. 9005-64-5)
polyoxyethylene(4) sorbitan monolaurate (Tween® 21, CAS No. 9005-64-5)
polyoxyethylene(4) sorbitan monostearate (Tween® 61, CAS No. 9005-67-8)
polyoxyethylene(20) sorbitan tristearate (Tween® 65, CAS No. 9005-71-4)
polyoxyethylene(20) sorbitan monooleate (Tween® 80, CAS No. 9005-65-6)
polyoxyethylene(5) sorbitan monooleate (Tween® 81, CAS No. 9005-65-5)
polyoxyethylene(20) sorbitan trioleate (Tween® 85, CAS No. 9005-70-3).
Polyoxyethylene(20) sorbitan monopalmitate (Tween® 40, CAS No. 9005-86-7) and
polyoxyethylene(20) sorbitan monostearate (Tween® 60, CAS No. 9005-67-8)
   are particularly advantageous.

The polysorbates are advantageously used in a concentration of from 0.1 to 5% by weight and in particular in a concentration of from 1.5 to 2.5% by weight, based on the total weight of the composition, individually or as a mixture of two or more polysorbates.

Further Conditioners

If desired, the compositions according to the invention comprise further conditioners in addition to the polymers to be used according to the invention. The further conditioners for the cosmetic compositions according to the invention chosen are preferably those conditioners which are described on page 34, line 24 to page 37, line 10 of WO 2006/106140. Reference is hereby made to the content of the specified passage in its entirety.

Rheology Modifiers

Suitable rheology modifiers are primarily thickeners. Thickeners suitable for shampoos and haircare compositions are given in “Kosmetik und Hygiene von Kopf bis Fuβ” [Cosmetics and Hygiene from Head to Toe], Ed. W. Umbach, 3^{rd} edition, Wiley-VCH, 2004, pp. 235-236, to which reference is made at this point in its entirety. Suitable thickeners for the cosmetic compositions according to the invention are described, for example, also on page 37, line 12 to page 38, line 8 of WO 2005/106140. Reference is hereby made to the content of the specified passage in its entirety.

Preservatives

The cosmetic compositions according to the invention can also comprise preservatives. Compositions with high water contents have to be reliably protected against the build-up of germs. Suitable preservatives for the cosmetic compositions according to the invention are described, for example, on page 38, line 10 to page 39, line 18 of WO 2005/108140. Reference is hereby made to the content of the specified passage in its entirety.

Complexing agents: since the raw materials and also the cosmetic compositions themselves are often produced primarily in steel apparatuses, the end products can comprise iron (ions) in trace amounts, in order to prevent these impurities adversely affecting the product quality through reactions with dyes and perfume oil constituents, complexing agents such as salts of ethylenediaminetetraacetic acid, of nitrilotriacetic acid, of iminodisuccinic acid or phosphates are added.

UV photoprotective filters: in order to stabilize the ingredients present in the cosmetic compositions according to the invention, such as, for example, dyes and perfume oils, against changes due to UV light, UV photoprotective filters, such as, for example, benzophenone derivatives, can be incorporated. Suitable UV photoprotective filters for the cosmetic compositions according to the invention are described, for example, on page 39, line 20 to page 41, line 10 of WO 2006/106140. Reference is hereby made to the content of the specified passage in its entirety.

Antioxidants: a content of antioxidants in the compositions according to the invention is generally preferred. According to the invention, antioxidants which can be used are all antioxidants customary or suitable for cosmetic applications. Suitable antioxidants for the cosmetic compositions according to the invention are described, for example, on page 41, line 12 to page 42, line 33 of WO 2006/108140. Reference is hereby made to the content of the specified passage in its entirety.

Buffers: buffers ensure the pH stability of the cosmetic compositions. Citrate, lactate and phosphate buffers are primarily used.

Solubility promoters: they are used in order to bring care oils or perfume oils clearly into solution and also to keep them clearly in solution at low temperature. The most common solubility promoters are ethoxylated nonionic surfactants, for example hydrogenated and ethoxylated ricinus oils.

Antimicrobial agents: furthermore, antimicrobial agents can also be used. These include, in general, all suitable preservatives with specific action against gram-positive bacteria, e.g. triclosan (2,4,4′-trichloro-2-hydroxy diphenyl ether), chlorhexidine (1,1′-hexamethylenebis[5-(4-chlorophenyl)biguanide), and TTC (3,4,4′-trichlorocarbanilide). Quaternary ammonium compounds are in principle likewise suitable and are preferably used for disinfectant soaps and washing lotions. Numerous fragrances also have antimicrobial properties. A large number of essential oils or their characteristic ingredients, such as, for example, clove oil (eugenol), mint oil (menthol) or thyme oil (thymol), also exhibit marked antimicrobial effectiveness.

The antibacterially effective substances are generally used in concentrations of from about 0.1 to 0.3% by weight.

Dispersants: if insoluble active ingredients, e.g. antidandruff active ingredients or silicone oils, are to be dispersed and kept permanently in suspension in the cosmetic compositions according to the invention, dispersants and thickeners, such as, for example, magnesium-aluminum silicates, bentonites, fatty acyl derivatives, polyvinylpyrrolidone or hydrocolloids, e.g. xanthan gum or carbomers, have to be used. According to the invention, preservatives are present in a total concentration of at most 2% by weight, preferably at most 1.5% by weight and particularly preferably at most 1% by weight, based on the total weight of the composition.

Apart from the abovementioned substances, the cosmetic compositions can, if appropriate, comprise the additives customary in cosmetics, for example perfume, dyes, refatting agents, complexing and sequestering agents, pearlizing agents, plant extracts, vitamins, active ingredients, pigments which have a coloring effect softening, moisturizing and/or humectant substances, or other customary constituents of a cosmetic or dermatological formulation, such as alcohols, polyols, polymers, organic acids for adjusting the pH, foam stabilizers, electrolytes, organic solvents or silicone derivatives.

With regard to the specified further ingredients known to the person skilled in the art for the compositions, reference may be made to “Kosmetik und Hygiene von Kopf bis Fuβ” [Cosmetics and Hygiene from Head to Toe], Ed. W. Umbach, 3^{rd} edition, Wiley-VCH, 2004, pp. 123-128, to which reference is made at this point in its entirety.

The polymers to be used according to the invention are, especially in cationic form, suitable for enhancing or for increasing the deposition amount and rate, and also the residence time of further active ingredients, such as, for example, silicones or UV photoprotective filters, on the skin and/or the hair. Substances or compositions which have such effects are also referred to as depositioning aids.

U.S. Pat. No. 6,998,113 describes rinse-off preparations which lead to the skin treated therewith being effectively protected from UV radiation. Some of the preparations described therein comprise cationic polymers. For the purposes of the present invention, the copolymers according to the invention can also be used in the preparations of U.S. Pat. No. 8,938,113. In particular, the polymers to be used according to the invention can be used for the purpose specified by U.S. Pat. No. 6,998,113 in sunscreen, washing and bathing preparations. Reference is hereby made to the disclosure of U.S. Pat. No. 6,998,113 in its entirety.

Suitable silicones are described, for example, in U.S. Pat. No. 5,935,561, column 13, I.64 to column 18, I.61, to which reference is hereby made in its entirety.

By way of representation, mention may be made of:
dimethicones
polyalkyl- or polyarylsiloxanes (U.S. Pat. No. 5,935,561, column 13, formula I)
alkylamino-substituted silicones (U.S. Pat. No. 5,935,561, column 14, formula II (amodimethicones))
cationic silicones (U.S. Pat. No. 5,935,561, columns 14 and 15, formula III)
trimethylsilylamodimethicones (U.S. Pat. No. 5,935,561, column 15, formula IV)
silicones as in U.S. Pat. No. 5,935,561, column 15, formula V
cyclic polysiloxanes as in U.S. Pat. No. 5,935,561, column 16, formula VI
ethoxylated glycerol fatty acid esters

The cosmetic compositions according to the invention, such as shampoos and haircare compositions, comprise, if appropriate, ethoxylated oils selected from the group of ethoxylated glycerol fatty acid esters, particularly preferably PEG-10 olive oil glycerides, PEG-11 avocado oil glycerides. PEG-11 cocoa butter glycerides, PEG-13 sunflower oil glycerides, PEG-15 glycerylisostearate, PEG-9 coconut fatty acid glycerides, PEG-54 hydrogenated ricinus oil. PEG-7 hydrogenated ricinus oil, PEG-60 hydrogenated ricinus oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm: kernel oil glycerides, PEG-35 ricinus oil, olive oil PEG-7 ester, PEG-6 caprylic acid/capric acid glycerides, PEG-10 olive oil glycerides, PEG-13 sunflower oil glycerides, PEG-7 hydrogenated ricinus oil, hydrogenated palm kernel oil glyceride PEG-6 ester, PEG-20 corn oil glycerides, PEG-18 glyceryl oleate cocoate, PEG-40 hydrogenated ricinus oil, PEG-40 ricinus oil, PEG-60 hydrogenated ricinus oil, PEG-60 corn oil glycerides, PEG-54 hydrogenated ricinus oil. PEG-45 palm kernel oil glycerides, PEG-80 glyceryl cocoate, PEG-60 almond oil glycerides, PEG-60 evening primrose glycerides, PEG-200 hydrogenated glyceryl palmate, PEG-90 glyceryl isostearate.

Preferred ethoxylated oils are PEG-7 glyceryl cocoate, PEG-9 coconut glycerides, PEG-40 hydrogenated ricinus oil, PEG-200 hydrogenated glyceryl palmate. Ethoxylated glycerol fatty acid esters are used in aqueous cleaning formulations for various purposes. Glycerol fatty acid esters with a degree of ethoxylation of about 30-50 serve as solubility promoters for nonpolar substances such as perfume oils. Highly ethoxylated glycerol fatty acid esters are used as thickeners.

Active Ingredients

Highly diverse active ingredients of varying solubility can be incorporated homogeneously into the cosmetic compositions according to the invention. Advantageous active ingredients in the cosmetic compositions according to the invention are described, for example, on page 44, line 24 to page 49, line 39 of WO 2006/106140. Reference is hereby made to the content of the specified passage in its entirety.

UV Photoprotective Agents

In a preferred embodiment, the compositions according to the invention comprise UV photoprotective agents for protecting the skin and/or the hair. Suitable UV photoprotective agents are described in detail in WO 2006/106114, p. 24, I.4 to p. 27, I.27, to which reference is hereby made in its entirety.

The cosmetic compositions advantageously comprise substances which absorb UV radiation in the UVB region and substances which absorb UV radiation in the UVA region, the total amount of the filter substances being, for example, 0.1 to 30% by weight, preferably 0.5 to 20% by weight, in particular 1 to 15% by weight, based on the total weight of the compositions, in order to provide cosmetic compositions which protect the skin from the entire range of ultraviolet radiation.

The majority of the photoprotective agents in the cosmetic or dermatological compositions serving to protect the human epidermis consists of compounds which absorb UV light in the UV-B region. For example, the fraction of the UV-A absorbers to be used according to the invention is 10 to 90% by weight, preferably 20 to 50% by weight, based on the total amount of substances absorbing UV-B and UV-A.

Pearlescent Waxes

Suitable pearlescent waxes for the cosmetic compositions according to the invention are described, for example, on page 50, line 1 to line 18 of WO 2006/106140. Reference is hereby made to the content of the specified passage in its entirety. The cosmetic compositions according to the invention can furthermore comprise glitter substances and/or other effect substances (e.g. color streaks).

Emulsifiers

In a preferred embodiment of the invention, the cosmetic compositions according to the invention are in the form of emulsions. Such emulsions are prepared by known methods. Suitable emulsifiers for the emulsions according to the invention are described, for example, on page 50, line 18 to page 53, line 4 of WO 2005/106140. Reference is hereby made to the content of the specified passage in its entirety.

Perfume Oils

If perfume oils are to be added to the cosmetic compositions according to the invention, then suitable perfume oils are described, for example, on page 53, line 10 to page 54, line 3 of WO 2008/108140. Reference is hereby made to the content of the specified passage in its entirety.

Pigments

If appropriate, the cosmetic compositions according to the invention furthermore comprise pigments. The pigments are present in the product mostly in undissolved form and may be present in an amount of from 0,01 to 25% by weight, particularly preferably from 5 to 15% by weight. The preferred particle size is 1 to 200 μm, in particular 3 to 150 μm, particularly preferably 10 to 100 μm.

Suitable pigments for the compositions according to the invention are described, for example on page 54, line 5 to page 55, line 19 of WO 2006/106140. Reference is hereby made to the content of the specified passage in its entirety.

Polymers

In a preferred embodiment, apart from the polymers to be used according to the invention produced by the method according to the invention, the cosmetic compositions according to the invention comprise further polymers. Suitable additional polymers for the compositions according to the invention are described, for example, on page 55, line 21 to page 63, line 2 of WO 2006/106140. Reference is hereby made to the content of the specified passage in its entirety.

Shampoo Types

A preferred embodiment of the invention are hair shampoos comprising the copolymers according to the invention. Additional requirements are, if appropriate, placed on shampoos according to hair quality or scalp problem. The mode of action of the preferred shampoo types with the most important additional effects or most important special objectives is described below.

According to the invention, preference is given, for example, to shampoos for normal or rapidly greasing or damaged hair, antidandruff shampoos, baby shampoos and two-in-one shampoos (i.e. shampoo and conditioner in one).

Shampoos according to the invention for normal hair: hair washing should free hair and scalp from the skin sebum formed in sebaceous glands, the inorganic salts emerging from sweat glands with water, amino acids, urea and lactic acid, shed skin particles, environmental dirt, odors and, if appropriate, residues of hair cosmetic treatments. Normal hair means short to shoulder-length hair which is only slightly damaged. Accordingly, the fraction of conditioning auxiliaries should be optimized to this hair type. Shampoos according to the invention for rapidly greasing hair: increased sebum production by the sebaceous glands on the scalp leads just 1-2 days after hair washing to a straggly, unsightly hairstyle. Oil- and wax-like skin sebum constituents weigh down the hair and reduce the friction from hair to hair and thus reduce the hairstyle hold. The actual hair cosmetic problem in the case of rapidly greasing hair is thus the premature collapse of voluminous hairstyles, in order to avoid this, it is necessary to prevent the hair surface becoming weighed down and too smooth and supple. This is preferably achieved through the surfactant base of highly cleaning washing raw materials that are characterized by particularly low substantively. Additional care substances which would add to the skin sebum, such as refatting substances, are used in shampoos for rapidly greasing hair only with the greatest of care, if at all. Volumizing shampoos according to the invention for fine hair can be formulated comparably.

Shampoos according to the invention for dry, stripped (damaged) hair; the structure of the hair is changed in the course of hair growth by mechanical influences such as combing, brushing and primarily back-combing (combing against the direction of growth), by the effect of UV radiation and visible light and by cosmetic treatments, such as permanent waves, bleaching or coloring. The flake layer of the hair has an increasingly stripped appearance from root to the end; in extreme cases, if is completely worn away at the end, and the hair ends are split (split ends). Damaged hair can in principle no longer be returned to the state of healthy hair regrowth. However, if is possible to come very close to this ideal state as regards feel, shine and combability by using shampoos according to the invention with, if appropriate, high fractions of care substances (conditioners).

An even better hair-conditioning effect than with a shampoo is achieved with a haircare composition according to the invention, for example in the form of a rinse or cure treatment after hair washing. Rinses or cures for hair which comprise polymers to be used according to the invention are likewise in accordance with the invention. 2-in-1 shampoos according to the invention are particularly high-care shampoos in which, as a result of the design as “shampoo and conditioner in one” the additional care benefit is placed equally alongside the basic cleaning benefit. 2-in-1 compositions according to the invention comprise increased amounts of conditioners.

Antidandruff shampoos: compared with antidandruff hair tonics, antidandruff shampoos according to the invention have the advantage that they not only reduce the formation of new visible flakes through appropriate active ingredients against dandruff attack and prevent such formation upon long-term application, but also remove flakes already shed with the hair washing. However, after rinsing out the wash liquor, only a small, but adequate amount of the active ingredients remains on the scalp and hair. There are various antidandruff active ingredients which can be incorporated into the shampoo compositions according to the invention, such as, for example, zinc pyrithion, ketoconazole, elubiol, clotrimazole, climbazole or piroctone olamine. Additionally, these substances have a normalizing effect on shedding.

The basis of antidandruff shampoos corresponds primarily to the formulation of shampoos for normal hair with a good cleaning effect.

Baby shampoos: in a preferred embodiment of the invention, the shampoo preparations according to the invention are baby shampoos. These are optimally skin- and mucosa-compatible. Combinations of washing raw materials with very good skin compatibility form the basis of these shampoos. Additional substances for further improving the skin and mucosa compatibility and the care properties are advantageously added, such as, for example, nonionic surfactants, protein hydrolyzates and panthenol or bisabolol. All of the required raw materials and auxiliaries, such as preservatives, perfume oils, dyes etc., are selected from the aspect of high compatibility and mildness.

Shampoos for dry scalp: in a further preferred embodiment of the invention, the shampoo preparations according to the invention are shampoos for dry scalp. The primary aim of these shampoos is to prevent the scalp from drying out since dry scalp can lead to irritation, reddening and inflammation. As also in the case of the baby shampoos, combinations of washing raw materials with very good skin compatibility form the basis of these shampoos. Additionally, if appropriate, refatting agents and humectants, such as, for example, glycerol or urea, can be used.

The shampoo compositions according to the invention can also be present as shampoo concentrates with increased surfactant contents of 20-30% by weight. They are based on special washing raw material combinations and consistency regulators which ensure good spreadability and the spontaneous foaming ability even of a small application amount. A particular advantage is, for example, the possibility of achieving the productivity of 200 ml of shampoo with a 100 ml bottle.

Supply Form

It is advantageous if the compositions according to the invention are stored in a bottle or squeezable bottle and are applied from this. Accordingly, bottles or squeezable bottles which comprise a composition according to the invention are also in accordance with the invention.

The copolymers according to the invention, as defined above, can preferably be used in shampoo formulations in particular as conditioners. Preferred shampoo formulations comprise

a) 0.05 to 10% by weight of at least one copolymer according to the invention,

b) 25 to 94.95% by weight of water,

c) 5 to 50% by weight of surfactants,

d) 0 to 5% by weight of a further conditioner,

e) 0 to 10% by weight of further cosmetic constituents.

All anionic, neutral, amphoteric or cationic surfactants customarily used in shampoos can be used in the shampoo formulations. Suitable surfactants have been specified above.

Soaps and Syndets

Further compositions according to the invention which comprise the copolymers according to the invention are, for example, soaps and syndets.

Soap is formed in the reaction of a (neutral) fat or fatly acids obtained therefrom or fatty acid methyl ester with sodium hydroxide or potassium hydroxide (saponification). Soap is chemically the alkali metal salt of fatty acids in the composition. The neutral fats usually used in the manufacture of soap are beef tallow or palm oil in a mixture with coconut oil or palm kernel oil and—more rarely—other natural oils or fats, the quality of the starting fats being highly influential on the quality of the soap obtained therefrom.

Of importance for selecting the fatty components is the distribution of the chain lengths of the corresponding fatty acids. Normally, especially C12-C18-fatty acids are in demand. Since laurate soap foams particularly well, laurin-rich coconut oil or similarly formulated palm kernel oil are usually used in relatively high fractions (up to 50% of the neutral fatty mixture) for soaps for which a large amount of foam during use is desired. The sodium salts of the specified fatty acid mixtures are solid, whereas the potassium salts are soft and pasty. For this reason, the hydroxide solution component used for producing solid soaps is preferably sodium hydroxide solution, and for liquid-pasty soaps is preferably potassium hydroxide solution. During the saponification, the ratio of hydroxide solution to fatty acid is selected so that, at most, a minimum excess of hydroxide solution (max. 0.05%) is present in the finished soap bar.

The soaps usually include toilet, curd, transparent, luxury, cream, freshening/deodorant, baby, skin protection, abrasive, floating and liquid soaps and also washing pastes and soap leaves.

Besides the polymers to be used according to the invention, soaps according to the invention advantageously furthermore comprise antioxidants, complexing agents and humectants, and, if appropriate, fragrances, dyes and further cosmetically acceptable ingredients. Such further suitable ingredients are specified above.

Syndets (synthetic detergents) are alternatives to conventional soaps which have certain advantages as a result of the varying composition compared to soap, whereas soap more likely has disadvantages.

Syndets comprise, as foam and cleaning components, washing-active substances (surfactants) which are obtained by chemical synthesis. By contrast, soaps are—as described—salts of naturally occurring fatty acids. For syndets, skin-mild, readily biodegradable surfactants are used, preferably fatty acid isethionates (sodium cocoyl isethionate), sulfosuccinic acid half-esters (disodium lauryl sulfosuccinate), alkyl polyglycosides (decyl glucoside), amphoteric surfactants (e.g. sodium cocoamphoacetate). In addition, monoglyceride sulfate and ether carboxylates sometimes play a role. Fatty alcohol sulfate (e.g. sodium lauryl sulfate) has largely lost its former significance as base surfactant for syndets. The base surfactants are combined with builder substances, retailing agents and further additives to give formulations which can be processed by customary soap technology and produce bars which behave as far as possible “soap-like”, but without the mentioned disadvantages of soap. They foam at every water hardness and have a very good cleaning power. Their pH can be adjusted within a wide range (mostly between 4 and 8). On account of the more intensive cleaning/degreasing power of the base surfactants, the surfactant fraction in the syndet is usually significantly lower, the fraction of superfatting agents is significantly higher than in soaps without the foaming ability being reduced. Syndets are recommended specifically for the cleansing of sensitive skin, of youthful-blemished skin and for face washing.

Alongside the (soap-free) syndets is also found the market segment of half- or combars (derived from combination bar). These are bars which comprise both soap and syndet surfactants. Combars comprise 10 to 80% by weight of soap. They represent a compromise between soaps and syndets for the criteria of costs, foaming ability, skin feel and compatibility. When washing with a combar, a pH of from about 7 to 9 is established, depending on its soap fraction.

As regards possible formulations for soaps and syndets known to the person skilled in the art, reference may be made to “Kosmetik und Hygiene von Kopf bis Fuβ” [Cosmetics and Hygiene from Head to Toe], Ed. W. Umbach, 3^{rd} edition, Wiley-VCH, 2004, pp. 112-122, to which reference is made at this point in its entirety.

Shower Bath and Bathing Products

As regards specific compositions for shower bath and bathing products or washing lotions, reference may be made to “Kosmetik und Hygiene von Kopf bis Fuβ” [Cosmetics and Hygiene from Head to Toe], Ed. W. Umbach, 3^{rd} edition, Wiley-VCH, 2004, pp. 128-134, to which reference is made at this point in its entirety.

The invention further provides the use of the polymers described above as auxiliaries in pharmacy, preferably as or in (a) coating compositions) for solid drug forms, for modifying rheological properties, as surface-active compound, as or in (an) adhesive(s), and as or in (a) coating compositions) for the textile, paper, printing and leather industries.

| | | | | | | AcA | | | | | | | |
|---|---|---|---|---|---|---|---|---|---|---|---|---|---|
| | | | | | | 10% | | | | | | | |
| | ImEMA | VP | AA | DADMAC | | in | EtOH | EA | | Initiator | | | |
| | 100% | 100% | 100% | (ml) 30% | H₂O | water | 100% | 100% | Initiator | amount | | T | |
| Polymer | (g) | (ml) | (ml) | in water | (ml) | (ml) | (ml) | (ml) | (20%) | (μl) | pH | (° C.) | |
| 1 | 14 | 0 | 0 | 0 | 5.5 | 50 | 0 | 0 | V50 | 250 | 6 | 65 | yellow polymer solution |
| | | | | | | | | | | | | | Mₙ = 215 000 g/mol M_{w} = 1 030 000 |
| | | | | | | | | | | | | | g/mol |
| 2 | 14 | 0 | 0 | 0 | 6 | 50 | 0 | 0 | V50 | 500 | 6 | 65 | yellow polymer solution |
| | | | | | | | | | | | | | Mₙ = 235 000 g/mol M_{w} = 820 000 g/mol |
| 3 | 7 | 6.7 | 0 | 0 | 6.3 | 50 | 0 | 0 | V50 | 500 | 5 | 65 | yellowish polymer solution |
| 4 | 7 | 0 | 6.7 | 0 | 56.3 | 0 | 0 | 0 | V50 | 500 | 4 | 65 | white polymer dispersion |
| 5 | 7 | 0 | 0 | 12 | 1 | 50 | 0 | 0 | V50 | 500 | 5 | 65 | yellowish polymer solution |
| | | | | | | | | | | | | | Mₙ = 66 000 g/mol M_{w} = 329 000 g/mol |
| 6 | 7 | 0 | 0 | 0 | 6 | 50 | 0 | 0 | V50 | 500 | 5 | 65 | white polymer dispersion |
| | | | | | | | | | | | | | Mn = 89 000 g/mol Mw = 385 000 g/mol |
| 7 | 14 | 0 | 0 | 0 | 0 | 0 | 55.5 | 0 | V50 | 500 | nd | 65 | yellowish polymer solution |
| | | | | | | | | | | | | | Mₙ = 52 000 g/mol M_{w} = 171 000 g/mol |
| 8 | 14 | 0 | 0 | 0 | 39 | 0 | 17 | 0 | V50 | 500 | 8 | 65 | yellowish polymer solution |
| | | | | | | | | | | | | | Mₙ = 87 000 g/mol M_{w} = 439 000 g/mol |
| 9 | 3.5 | 0 | 10 | 0 | 0 | 0 | 0 | 56 | tBPPiv | 500 | nd | 75 | white precipitation polymer |

| | | | | | | | | | | Kempf | |
|---|---|---|---|---|---|---|---|---|---|---|---|
| | VP | MAM | ImEMA | | | | | | Stiffening | tackiness | FS |
| Sample | wt-% | wt-% | wt-% | K value | Appearance | Viscosity | pH | Structure | effect | 20° C./80% r.h. | [cN] |
| 10 | 57 | 40 | 3 | 59.8 | blue-tinged | 43400 | 7.1 | 2 | good | 2 | 140 |
| 11 | 57 | 40 | 3 | 70.4 | blue-tinged | 45800 | 7.1 | 2-3 | very good | 2 | 193 |
| 12 | 55.5 | 69 | 5.5 | 61.2 | blue-tinged | 43600 | 7.2 | 2-3 | very good | 2 | 181 |
| C1 | 57 | 40 | VI: 3 | 60 | clear | 20300 | 6.9 | 1 | good | 0-1 | 98 |

### EXAMPLES

The following examples are intended to explain the invention in greater detail without limiting it thereto.

Abbreviations

AA Acrylic acid

DADMAC Diallyldimethylammonium chloride

EA Ethyl acetate

AcA Acetic acid

EtOH Ethanol

ImEMA 2-(1-imidazolyl)ethyl methacrylate

MAM Methylmethacrylamide

tBPPiv tert-Butyl perpivalate

Wako® V50 2,2′-azobis(2-amidinopropane)dihydrochloride

VP N-Vinylpyrrolidone

VI N-Vinylimidazole

Polymers 1 to 3 in Table 2 above were prepared in a batch procedure in a Workstation Model A100 from Chemspeed® Technologies. The reaction mixture was about 70 ml in each case.

ImEMA/DADMAC Copolymer (Polymer 5)

To produce a P(ImEMA-co-DADMAC) copolymer, 7 g of ImEMA and 12 ml of a 30% strength by weight aqueous DADMAC solution were mixed, in addition, 50 ml of 10% strength by weight acetic acid and 1 ml of water were added. 500 ml of a 20% strength by weight aqueous Wako® V50 initiator solution were added and the reaction mixture was heated to 65° C. and polymerized for 3 hours. The resulting copolymer had a number-average molecular weight determined by size exclusion chromatography (calibration using narrow-distribution pulluian (=linear polymaltotriose) standard (PBS, Germany) with molecular weights of Mₙ=5800 to Mₙ=710 000 g/mol, and also maltohexaose (Mₙ=992 g/mol); elution areas lying outside of this interval were estimated by extrapolation), of Mₙ=66 000 g/mol and a mass-average molecular weight of M_{w}=329 000 g/mol.

Copolymer VP/MAM/ImEMA (Polymer 10)

To produce a P(VP-co-MAM-co-ImEMA) copolymer, the initial charge (55.19 g of demineralized water, 7 g of VP, 16.01 g of feed 1, 1.04 g of feed 2) was gassed with nitrogen and heated to an internal reactor temperature of 70° C. After reaching the internal temperature, feed 1 (286.67 g of MAM, 0.35 g of phosphoric acid, 3.19 g of ImEMA, 50 g of VP) and 2 (0.8 g of Wako V50, 20 g of demineralized water) were started. Feed 1 was metered in over 4 hours, and feed 2 was metered in over 4.5 hours. When the feeds were complete, the mixture was further polymerized for a further 2 hours. A colorless, slightly opaque polymer solution is obtained. Polymers 11, 12 and C1 in table 2 were prepared analogously.

Determination of the K Value

The K values were measured in accordance with Fikentscher, Cellulosechemie [Cellulose Chemistry], Vol. 13, pp. 58 to 64 (1932) at 25° C. in aqueous solution and are a measure of the molecular weight. The aqueous solution of the polymers comprises 1 g of polymer in 100 ml of solution.

The K value is measured in a micro-Ubbelohde capillary model M Ic from Schott.

Tackiness:

The tackiness of the polymer films was determined in accordance with the method described in Chemie in unserer Zeit 36 (2002) pages 44-52.

Testing the Flexural Strength:

The tests were carried out in a climatically controlled room at 20° C. and 85% relative humidify using a tensile/pressure testing instrument (model Easytest 88 802, Frank). The hair tress was placed symmetrically on two cylindrical rollers (diameter 4 mm, gap=90 mm) of the sample holder. The tress was then bent exactly in the middle from above using a rounded punch by about 40 mm (until the gel film breaks). The force required for this was measured using a load cell (50 N) and given in Newtons.

Examples of Cosmetic Compositions

Hair Cosmetic Composition (General)

a) 0.01 to 5% by weight of a polymer used according to the invention

b) 25 to 99.89% by weight of water and/or alcohol

c) 0 to 95.99% by weight of further constituents

Alcohol is to be understood as meaning all alcohols customary in cosmetics, for example ethanol, isopropanol, n-propanol.

Further constituents are to be understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein-hydrolysates, alpha- and beta-hydroxycarboxylic acids, chitosans, protein hydrolysates, cosmetic polymers, stabilizers, pH regulators, dyes, viscosity regulators, gel formers, dyes, salts, humectants, refatting agents, complexing agents and further customary additives.

Shampoo Formulation/Shower Gel Formulation

Preferred shampoo formulations or shower gel formulations comprise

a) 0.01 to 5% by weight of a polymer used according to the invention

b) 25 to 99.99% by weight of water

c) 0-5% by weight of a further conditioner

d) 0-30% by weight of further cosmetic constituents

In addition, ail anionic, neutral, amphoteric or cationic surfactants customarily used in shampoos can be used in the shampoo formulations with the above provisos.

### Example 1

### Conditioner Shampoo with PQ-10

| | | |
|---|---|---|
| 35.70 | g | sodium laureth sulfate |
| 6.50 | g | cocamidopropylbetaine |
| 0.20 | g | polymer 5 |
| 0.40 | g | polyquaternium-10 |
| 0.10 | g | preservative |
| 0.10 | g | perfume oil/essential oil |
| ad 100 | g | aqua dem. |

Good conditioner shampoos are also obtained if, instead of the polymer according to Example 5, one or more of the polymers 1 to 4 or 6 to 12 are used.

### Example 2

### Conditioner Shampoo with GHTC

| | | |
|---|---|---|
| 35.70 | g | sodium laureth sulfate |
| 6.50 | g | cocamidopropylbetaine |
| 0.50 | g | polymer 5 |
| 0.20 | g | guar hydroxypropyltrimonium chloride |
| 0.10 | g | preservative |
| 0.10 | g | perfume oil/essential oil |
| ad 100 | g | aqua dem. |

Good conditioner shampoos are also obtained if, instead of the polymer according to Example 5, one or more of the polymers 1 to 4 or 6 to 12 are used,

### Example 3

### Conditioner Shampoo with Polyquaternium

| | | |
|---|---|---|
| 35.70 | g | sodium laureth sulfate |
| 6.50 | g | cocamidopropylbetaine |
| 0.20 | g | polymer 5 |
| 0.30 | g | polyquaternium-44 or polyquaternium-67 |
| 0.10 | g | preservative |
| 0.10 | g | perfume oil/essential oil |
| ad 100 | g | aqua dem. |

Good conditioner shampoos are also obtained if, instead of the polymer according to Example 5, one or more of the polymers 1 to 4 or 6 to 12 are used.

### Example 4

### Shampoo

Phase A

| | |
|---|---|
| 15.00 g | cocamidopropylbetaine |
| 10.00 g | disodium cocoamphodiacetate |
| 5.00 g | polysorbate 20 |
| 5.00 g | decyl glucoside |
| 0.20 g | polymer 5 |
| 0.10 g | perfume oil/essential oil |
| q.s. | preservative |
| 2.00 g | laureth-3 |
| ad 100 | aqua dem. |
| q.s. | citric acid |

Phase B

| | |
|---|---|
| 3.00 g | PEG-150 distearate |

Preparation

Weigh in components of phase A and dissolve; adjust pH to 6-7. Add phase B and heat to 50° C. Allow to cool to room temperature with stirring.

Good shampoos are also obtained if, instead of the polymer according to Example 5, one or more of the polymers 1 to 4 or 8 to 12 are used.

### Example 5

### Shampoo

| | |
|---|---|
| 30.00 g | sodium laureth sulfate |
| 6.00 g | sodium cocoamphoacetate |
| 0.50 g | polymer 5 |
| 3.00 g | sodium laureth sulfate, glycol distearate, cocamide |
| | MEA, laureth-10 |
| 2.00 g | dimethicone |
| q.s. | perfume |
| q.s. | preservative |
| q.s. | citric acid |
| 1.00 g | sodium chloride |
| ad 100 | aqua dem. |

Good shampoos are also obtained if, instead of the polymer according to Example 5, one or more of the polymers 1 to 4 or 6 to 12 are used.

### Example 6

### Shower Gel

| | |
|---|---|
| 20.00 g | ammonium laureth sulfate |
| 15.00 g | ammonium lauryl sulfate |
| 0.50 g | polymer 5 |
| 0.50 g | polyquaternium-7 |
| 2.50 g | sodium laureth sulfate, glycol distearate, cocamide |
| | MEA, laureth-10 |
| 0.10 g | perfume oil/essential oil |
| q.s. | preservative |
| 0.50 g | sodium chloride |
| ad 100 | aqua dem. |

Good shampoos are also obtained if, instead of the polymer according to Example 5, one or more of the polymers 1 to 4 or 8 to 12 are used.

### Example 7

### Shower Gel

| | |
|---|---|
| 40.00 g | sodium laureth sulfate |
| 5.00 g | decyl glucoside |
| 5.00 g | polymer 5 |
| 1.00 g | panthenol |
| 0.10 g | perfume oil/essential oil |
| q.s. | preservative |
| q.s. | citric acid |
| 2.00 g | sodium chloride |
| ad 100 | aqua dem. |

Good shower gels are also obtained if, instead of the polymer according to Example 5, one or more of the polymers 1 to 4 or 6 to 12 are used.

### Example 8

### Shampoo

| | |
|---|---|
| 12.00 g | sodium laureth sulfate |
| 1.50 g | decyl glucoside |
| 0.50 g | polymer 5 |
| 5.00 g | cocoglucoside glyceryl oleate |
| 2.00 g | sodium laureth sulfate, glycol distearate, cocamide |
| | MEA, laureth-10 |
| q.s. | preservative |
| q.s. | Sunset Yellow C.I. 15 985 |
| 0.10 g | perfume oil/essential oil |
| 1.00 g | sodium chloride |
| ad 100 | aqua dem. |

Good shampoos are also obtained if, instead of the polymer according to Example 5, one or more of the polymers 1 to 4 or 8 to 12 are used.

The polymers according to the invention are also used in hairstyling preparations, in particular hair foams (aerosol foams with propellant gas and pump foams without propellant gas), hairsprays (pump sprays without propellant gas) and hair gels.

Propellants are the customarily used propellants. Preference is given to mixtures of propane/butane, pentane, dimethyl ether, 1,1-difluoroethane (HFC-152 a), carbon dioxide, nitrogen or compressed air.

Aerosol Hair Foam

a) 0.1 to 10% by weight of a cosmetics polymer

b) 55 to 99.8% by weight of water and/or alcohol

c) 5 to 20% by weight of a propellant

d) 0.1 to 5% by weight of a polymer used according to the invention

e) 0 to 10% by weight of further constituents

Further constituents which may be used are, inter alia, ail emulsifiers customarily used in hair foams. Suitable emulsifiers may be nonionic, cationic or anionic or amphoteric.

Examples of nonionic emulsifiers (INCl nomenclature) are laureths, e.g. laureth-4; ceteths, e.g. cetheth-1, polyethylene glycol cetyl ether; ceteareths, e.g. cetheareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCl).

Anionic emulsifiers may, for example, be selected from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:

### Example 9

### Aerosol Fair Foam

| | |
|---|---|
| 2.00 g | cocotrimonium methosulfate |
| 0.10 g | perfume oil/essential oil |
| 3.50 g | setting polymer e.g. polyquaternium-46, PQ-44, |
| | VP/methacrylamide/vinyl |
| | imidazole copolymer, etc. |
| 0.80 g | polymer 3 |
| q.s. | preservative |
| 75.00 g | water dem. |
| 10.00 g | propane/butane (3.5 bar) |

Good aerosol hair foams are also obtained if, instead of the polymer according to Example 3, one or more of the polymers 1, 2 or 4 to 12 are used.

Styling Gel

a) 0.1 to 10% by weight of a cosmetics polymer

b) 60 to 99.85% by weight of water and/or alcohol

c) 0.05 to 10% by weight of a gel former

d) 0.1 to 5% by weight of a polymer according to the invention

e) 0 to 20% by weight of further constituents

Gel formers which can be used are ail gel formers customary in cosmetics. These include lightly crosslinked polyacrylic acid, for example carbomer (INCl), cellulose derivatives, e.g. hydroxypropylcellulose, hydroxyethylcellulose, cationically modified celluloses, polysaccharides, e.g. xanthan gum, caprylic/capric triglycerides, sodium acrylates copolymer, polyquaternium-32 (and) paraffinum liquidum (INCl), sodium acrylates copolymer (and) paraffinum liquidum (and) PPG-1 trideceth-6, acrylamidopropyltrimonium chloride/acrylamide copolymer, steareth-10 allyl ether acrylates copolymer, polyquaternium-37 (and) paraffinum liquidum (and) PPG-1 trideceth-6, polyquaternium-37 (and) propylene glycol dicaprate dicaprylate (and) PPG-1 trideceth-8, polyquaternium-7, polyquaternium-44, polyquaternium-67.

Good styling gels are also obtained if, instead of the polymer according to Example 3, one or more of the polymers 1, 2 or 4 to 12 are used.

### Example 10

### Hair Styling Gel

Phase A

| | |
|---|---|
| 0.50 g | carbomer or acrylates/C10-30 alkyl acrylate crosspolymer |
| 86.40 g | water dem. |

Phase B

| | |
|---|---|
| 0.70 g | triethanolamine |

Phase C

| | |
|---|---|
| 6.00 g | setting polymer e.g. VP/methacrylamide/vinyl imidazole |
| | copolymer |
| 5.00 g | PVP |
| 0.20 g | PEG-25 PABA |
| 0.50 g | polymer 3 |
| 0.10 g | perfume oil/essential oil |
| q.s. | PEG-14 dimethicone |
| q.s. | preservative |
| 0.10 g | tocopheryl acetate |

Good styling gels are also obtained if, instead of the polymer according to Example 3, one or more of the polymers 1, 2 or 4 to 12 are used.

### Example 11

### Hair Styling Gel

Phase A

| | |
|---|---|
| 0.50 g | carbomer or acrylates/C10-30 alkyl acrylate crosspolymer |
| 91.20 g | water dem. |

Phase B

| | |
|---|---|
| 0.90 g | tetrahydroxypropyl ethylenediamine |

Phase C

| | |
|---|---|
| 7.00 g | VP/VA copolymer |
| 0.40 g | polymer 3 |
| 0.20 g | perfume oil/essential oil |
| q.s. | preservative |
| 0.10 g | propylene glycol |

Good styling gels are also obtained if, instead of the polymer according to Example 3, one or more of the polymers 1, 2 or 4 to 12 are used.

| | |
|---|---|
| 6.00 g | caprylic/capric triglycerides |
| 3.00 g | glceryl stearate |
| 2.00 g | cetyl alcohol |
| 3.50 g | polymer 3 |
| 0.50 g | Cremophor A6 |
| 0.70 g | Cremophor A25 |
| 0.50 g | dimethicone |
| 0.50 g | vitamin E acetate |
| 2.00 g | caprylic/capric triglyceride and |
| | sodium acrylates copolymer |
| 1.00 g | D-panthenol USP |
| 0.10 g | EDTA |
| 10.00 g | setting polymer |
| q.s. | preservative |
| ad 100 g | water dem. |

Good hair wax creams are also obtained if, instead of the polymer according to Example 3, one or more of the polymers 1, 2 or 4 to 12 are used.

### Example 13

### Hair Pudding

| | |
|---|---|
| 3.00 g | Kollicoat IR (BASF) |
| q.s. | preservative |
| 2.00 g | setting polymer |
| 4.00 g | acrylates/beheneth-25 methacrylate copolymer |
| 0.70 g | polymer 3 |
| 0.50 g | dimethicone copolyol |
| 0.10 g | EDTA |
| 0.20 g | benzophenone-4 |
| ad 100 g | water dem. |

Good hair puddings are also obtained if, instead of the polymer according to Example 3, one or more of the polymers 1, 2 or 4 to 12 are used.

### Example 14

### Spray Gel

Phase A

| | |
|---|---|
| 1.25 g | setting polymer |
| 96.15 g | aqua dem. |

Phase B

| | |
|---|---|
| 0.70 g | acrylates/steareth-20 itaconate copolymer |
| 0.10 g | propylene glycol |
| 0.50 g | polymer 3 |
| 0.10 g | glycerol |
| 0.10 g | perfume oil/essential oil |
| q.s. | preservative |

Phase C

| | |
|---|---|
| 0.70 g | triethanolamine |

Good spray gels are also obtained if, instead of the polymer according to Example 3, one or more of the polymers 1, 2 or 4 to 12 are used.

A preparation suitable according to the invention for styling sprays can, for example, have the following composition;

### Example 15

### Pump Hairspray

| | |
|---|---|
| 11.20 g | PEG/PPG-25/25 dimethicone/acrylates copolymer |
| 2.80 g | VP/VA copolymer |
| 1.34 g | aminomethylpropanol |
| 0.30 g | polymer 3 |
| 0.10 g | perfume oil/essential oil |
| 11.26 g | aqua dem. |
| 73.00 g | alcohol |

Good pump hairsprays are also obtained if, instead of the polymer according to Example 3, one or more of the polymers 1, 2 or 4 to 12 are used.

### Example 16

### Pump Hairspray VOC55

| | |
|---|---|
| 2.00 g | VP/methacrylamide/vinyl imidazole copolymer |
| 1.90 g | polyquaternium-46 |
| 2.00 g | polymer 3 |
| 0.10 g | perfume oil/essential oil |
| 55.00 g | alcohol |
| 39.00 g | aqua dem. |

Good pump hairsprays VOC 55 are also obtained if, instead of the polymer according to Example 3, one or more of the polymers 1, 2 or 4 to 12 are used.

Skin Cosmetic Compositions

### Example 17

### Liquid Make-Up

Phase A

| | |
|---|---|
| 1.70 g | glyceryl stearate |
| 1.70 g | cetyl alcohol |
| 1.70 g | ceteareth-6 |
| 1.70 g | ceteareth-25 |
| 5.20 g | caprylic/capric triglyceride |
| 5.20 g | mineral oil or Luvitol ® Lite |
| | (INCI hydrogenated polyisobutene) |

Phase B

| | |
|---|---|
| q.s. | preservative |
| 4.30 g | propylene glycol |
| 2.50 g | polymer 3 |
| 59.50 g | aqua dem. |

Phase C

| | |
|---|---|
| 0.10 g | perfume oil/essential oil |

Phase D

| | |
|---|---|
| 2.00 g | iron oxides |
| 12.00 g | titanium dioxide |

Good liquid make-ups are also obtained if, instead of the polymer according to Example 3, one or more of the polymers 1, 2 or 4 to 12 are used.

### Example 18

### Eyeliner

Phase A

| | |
|---|---|
| 40.60 g | dist water |
| 0.20 g | disodium EDTA |
| q.s. | preservative |

Phase B

| | |
|---|---|
| 0.60 g | xanthan gum |
| 0.40 g | veegum |
| 3.00 g | butylene glycol |
| 0.20 g | polysorbate-20 |

Phase C

| | |
|---|---|
| 15.00 g | iron oxide/Al powder/silica (e.g. Sicopearl ® |
| | Fantastico Gold from BASF) |

Phase D

| | |
|---|---|
| 10.00 g | aqua dem. |
| 25.00 g | setting polymer (e.g. polyurethane-1 or |
| | VP/methacrylamide/vinyl imidazole |
| | copolymer, etc.) |
| 5.00 g | polymer 3 |

Good eyeliners are also obtained if, instead of the polymer according to Example 3, one or more of the polymers 1, 2 or 4 to 12 are used.

### Example 19

### Sunscreen Gel

Phase A

| | |
|---|---|
| 0.90 g | polymer 3 |
| 8.00 g | octyl methoxycinnamate |
| 5.00 g | octocrylene |
| 0.80 g | octyltriazone |
| 2.00 g | butylmethoxydibenzoylmethane |
| 2.00 g | tocopheryl acetate |
| 0.10 g | perfume oil/essential oil |

Phase B

| | |
|---|---|
| 0.30 g | acrylates/C10-30 alkyl acrylate crosspolymer |
| 0.20 g | carbomer |
| 5.00 g | glycerol |
| 0.20 g | disodium EDTA |
| q.s. | preservative |
| 75.30 g | aqua dem. |

Phase C

| | |
|---|---|
| 0.20 g | sodium hydroxide |

Good sunscreen gels are also obtained if, instead of the polymer according to Example 3, one or more of the polymers 1, 2 or 4 to 12 are used.

### Example 20

### Sunscreen Emulsion with TiO₂ and ZnO₂

Phase A

| | |
|---|---|
| 1.00 g | PEG-7 hydrogenated castor oil |
| 5.00 g | polymer 3 |
| 2.00 g | PEG-45/dodecyl glycol copolymer |
| 3.00 g | isopropyl myristate |
| 7.90 g | jojoba (Buxus Chinensis) oil |
| 4.00 g | octyl methoxycinnamate |
| 2.00 g | 4-methylbenzylidenecamphor |
| 3.00 g | titanium dioxide, dimethicone |
| 1.00 g | dimethicone |
| 5.00 g | zinc oxide, dimethicone |

Phase B

| | |
|---|---|
| 0.20 g | disodium EDTA |
| 5.00 g | glycerol |
| q.s. | preservative |
| 60.80 g | aqua dem. |

Phase C

| | |
|---|---|
| 0.10 g | perfume oil/essential oil |

Good sunscreen emulsions are also obtained if, instead of the polymer according to Example 3, one or more of the polymers 1, 2 or 4 to 12 are used.

### Example 21

### Face Tonic

Phase A

| | |
|---|---|
| 3.00 g | polymer 3 |
| 0.10 g | perfume oil/essential oil |
| 0.30 g | bisabolol |

Phase B

| | |
|---|---|
| 3.00 g | glycerol |
| 1.00 g | hydroxyethyl cetyldimonium phosphate |
| 5.00 g | witch hazel (Hamameiis Virginiana) distillate |
| 0.50 g | panthenol |
| q.s. | preservative |
| 87.60 g | aqua dem. |

Good face tonics are also obtained if, instead of the polymer according to Example 3, one or more of the polymers 1, 2 or 4 to 12 are used.

### Example 22

### Face Washing Paste with Peeling Effect

Phase A

| | |
|---|---|
| 73.00 g | aqua dem. |
| 1.50 g | carbomer |
| q.s. | preservative |

Phase B

| | |
|---|---|
| q.s. | perfume oil |
| 7.00 g | potassium cocoyl hydrolyzed protein |
| 4.00 g | polymer 3 |

Phase C

| | |
|---|---|
| 1.50 g | triethanolamine |

Phase D

| | |
|---|---|
| 13.00 g | polyethylene (Luwax A ™ from BASF) |

Good face washing pastes are also obtained if, instead of the polymer according to Example 3, one or more of the polymers 1, 2 or 4 to 12 are used.

### Example 23

### Soap

Phase A

| | |
|---|---|
| 25.00 g | potassium cocoate |
| 20.00 g | disodium cocoamphodiacetate |
| 2.00 g | lauramide DEA |
| 1.0 g | glycol stearate |
| 2.00 g | polymer 3 |
| 50.00 g | aqua dem. |
| q.s. | citric acid |

Phase B

| | |
|---|---|
| q.s. | preservative |
| 0.10 g | perfume oil/essential oil |

Good soaps are also obtained if, instead of the polymer according to Example 3, one or more of the polymers 1, 2 or 4 to 12 are used.

### Example 24

### Face Cleansing Milk O/W Type

Phase A

| | |
|---|---|
| 1.50 g | ceteareth-6 |
| 1.50 g | ceteareth-25 |
| 2.00 g | glyceryl stearate |
| 2.00 g | cetyl alcohol |
| 10.00 g | mineral oil |

Phase B

| | |
|---|---|
| 5.00 g | propylene glycol |
| q.s. | preservative |
| 1.00 g | polymer 3 |
| 66.30 g | aqua dem. |

Phase C

| | |
|---|---|
| 0.20 g | carbomer |
| 10.00 g | cetearyl octanoate |

Phase D

| | |
|---|---|
| 0.40 g | tetrahydroxypropylethylenediamine |

Phase E

| | |
|---|---|
| 0.10 g | perfume oil/essential oil |
| 0.10 g | bisabolol |

Good face cleansing milks are also obtained if, instead of the polymer according to Example 3, one or more of tie polymers 1, 2 or 4 to 12 are used.

### Example 25

### Transparent Soap

| | |
|---|---|
| 4.20 g | sodium hydroxide |
| 3.60 g | dist. water |
| 10.00 g | polymer 3 |
| 22.60 g | propylene glycol |
| 18.70 g | glycerol |
| 5.20 g | cocoamide DEA |
| 2.40 g | cocamine oxide |
| 4.20 g | sodium lauryl sulfate |
| 7.30 g | myristic acid |
| 16.60 g | stearic acid |
| 5.20 g | tocopherol |

Good transparent soaps are also obtained if, instead of the polymer according to Example 3, one or more of the polymers 1, 2 or 4 to 12 are used.

### Example 26

### Shaving Foam

| | |
|---|---|
| 6.00 g | ceteareth-25 |
| 5.00 g | poloxamer 407 |
| 52.00 g | aqua dem. |
| 1.00 g | triethanolamine |
| 5.00 g | propylene glycol |
| 1.00 g | PEG-75 lanolin oil |
| 5.00 g | polymer 3 |
| q.s. | preservative |
| 0.10 g | perfume oil/essential oil |
| 25.00 g | sodium laureth sulfate |

Bottling: 90 parts of active substance and 10 parts of propane/butane mixture 25:75.

Good shaving foams are-also obtained if, instead of the polymer according to Example 3, one or more of the polymers 1, 2 or 4 to 12 are used.

### Example 27

### Aftershave Balm

Phase A

| | |
|---|---|
| 0.25 g | acrylates/C10-30 alkyl acrylate crosspolymer |
| 1.50 g | tocopheryl acetate |
| 0.20 g | bisabolol |
| 10.00 g | caprylic/capric triglyceride |
| q.s. | perfume |
| 1.00 g | polymer 3 |

Phase B

| | |
|---|---|
| 1.00 g | panthenol |
| 15.00 g | alcohol |
| 5.00 g | glycerol |
| 0.05 g | hydroxyethylcellulose |
| 1.90 g | polymer 3 |
| 64.02 g | dist. water |

Phase C

| | |
|---|---|
| 0.08 g | sodium hydroxide |

Good aftershave balms are also obtained if, instead of the polymer according to Example 3, one or more of the polymers 1, 2 or 4 to 12 are used.

### Example 28

### Care Cream

Phase A

| | |
|---|---|
| 2.00 g | ceteareth-6 |
| 2.00 g | ceteareth-25 |
| 2.00 g | cetearyl alcohol |
| 3.00 g | glyceryl stearate SE |
| 5.00 g | mineral oil |
| 4.00 g | jojoba (Buxus Chinensis) oil |
| 3.00 g | cetearyl octanoate |
| 1.00 g | dimethicone |
| 3.00 g | mineral oil, lanolin alcohol |

Phase B

| | |
|---|---|
| 5.00 g | propylene glycol |
| 0.50 g | veegum |
| 1.00 g | panthenol |
| 1.70 g | polymer 3 |
| 6.00 g | polyquaternium-44 |
| q.s. | preservative |
| 60.80 g | aqua dem. |

Phase C

| | |
|---|---|
| q.s. | perfume |

Good care creams are also obtained if, instead of the polymer according to Example 3, one or more of the polymers 1, 2 or 4 to 12 are used.

Oral and Dental Care Preparations

### Example 29

### Toothpaste

Phase A

| | |
|---|---|
| 34.79 g | aqua dem. |
| 3.00 g | polymer 3 |
| 20.00 g | glycerol |
| 0.76 g | sodium monofluorophosphate |

Phase B

| | |
|---|---|
| 1.20 g | sodium carboxymethylcellulose |

Phase C

| | |
|---|---|
| 0.80 g | aroma oil |
| 0.06 g | saccharin |
| q.s. | preservative |
| 0.05 g | bisabolol |
| 1.00 g | panthenol |
| 0.50 g | tocopheryl acetate |
| 2.80 g | silica |
| 1.00 g | sodium lauryl sulfate |
| 7.90 g | dicalcium phosphate anhydrate |
| 25.29 g | dicalcium phosphate dihydrate |
| 0.45 g | titanium dioxide |

Good toothpastes are also obtained if, instead of the polymer according to Example 3, one or more of the polymers 1, 2 or 4 to 12 are used.

### Example 30

### Mouthwash

Phase A

| | |
|---|---|
| 2.00 g | aroma oil |
| 4.50 g | polymer 3 |
| 1.00 g | bisabolol |
| 30.00 g | alcohol |

Phase B

| | |
|---|---|
| 0.20 g | saccharin |
| 5.00 g | glycerol |
| q.s. | preservative |
| 5.00 g | poloxamer 407 |
| 52.30 g | aqua dem. |

Good mouthwashes are also obtained if, instead of the polymer according to Example 3, one or more of the polymers 1, 2 or 4 to 12 are used.

### Example 37

### Prosthesis Adhesive

Phase A

| | |
|---|---|
| 0.20 g | bisabolol |
| 1.00 g | beta-carotene |
| q.s. | aroma oil |
| 20.00 g | cetearyl octanoate |
| 5.00 g | silica |
| 33.80 g | mineral oil |

Phase B

| | |
|---|---|
| 5.00 g | polymer 3 |
| 35.00 g | PVP (20% strength solution in water) |

Good prosthesis adhesives are also obtained if, instead of the polymer according to Example 3, one or more of the polymers 1, 2 or 4 to 12 are used.

### Example 38

### Liquid Soap

| | |
|---|---|
| 15.0 g | coconut fatty acid, potassium salt |
| 3.0 g | potassium oleate |
| 5.0 g | Luvitol ® Lite (BASF) |
| 2.0 g | polymer of vinylpyrrolidone/stearyl methacrylate 70/30% |
| | by weight (K value 47; 1% in isopropanol) |
| 1.0 g | glycerol stearate |
| 0.5 g | polymer 3 |
| 2.0 g | ethylene glycol distearate |
| ad 100 | specific additives, complexing agents, fragrances, water |

Good liquid soaps are also obtained if, instead of the polymer according to Example 3, one or more of the polymers 1, 2 or 4 to 12 are used.

### Examples 39-41

### Conditioning Shampoo with Pearlescence

Data in % by weight

| Additive | Ex. 39 | Ex. 40 | Ex. 41 |
|---|---|---|---|
| Polymer 5 | 0.5 | 0.5 | 0.5 |
| Sodium laureth sulfate | 9.0 | 9.0 | 9.0 |
| Cocoamidopropylbetaine | 2.5 | 2.5 | 2.5 |
| Benzophenone-3 | 1.5 | 0.5 | 1.00 |
| Pearlizing agent | 2.0 | 2.0 | 2.0 |
| Luvitol Lite ® (BASF) | 0.1 | 0.15 | 0.05 |
| Disodium EDTA | 0.1 | 0.2 | 0.15 |
| Preservative, perfume, thickener, pH | q.s. | q.s. | q.s. |
| adjustment and solubility promoter | | | |
| Water | ad 100.0 | ad 100.0 | ad 100.0 |

The pH is adjusted to 6

Good conditioning shampoos with pearlescence are also obtained if, instead of the polymer according to Example 5, one or more of the polymers 1 to 4 or 8 to 12 are used.

### Examples 42-48

### Formulations for Showering, Washing, Bathing

Data in % by weight

| Additive | Ex. 42 | Ex. 43 | Ex. 44 | Ex. 45 | Ex. 46 |
|---|---|---|---|---|---|
| Texapon N 70 | 13.00 | 15.00 | 10.50 | 12.50 | 10.00 |
| Dehyton PK 45 | 7.50 | 7.00 | 5.00 | 5.50 | 10.00 |
| Cetiol HE | 2.00 | 2.50 | 3.50 | 5.00 | 2.30 |
| Perfume | 0.10 | 0.10 | 0.10 | 0.10 | 0.10 |
| Luvitol ® Lite (BASF) | 1.00 | 4.50 | 7.00 | 1.40 | 3.00 |
| D-Panthenol USP | 1.00 | 1.50 | 1.80 | 1.70 | 1.40 |
| Preservative | 0.10 | 0.10 | 0.10 | 0.10 | 0.10 |
| Citric acid | 0.10 | 0.10 | 0.10 | 0.10 | 0.10 |
| Polymer 3 | 0.50 | 1.00 | 0.50 | 0.20 | 0.10 |
| Sodium chloride | 1.50 | 1.40 | 1.40 | 1.30 | 1.50 |
| Water dem. | ad 100 | ad 100 | ad 100 | ad 100 | ad 100 |

Good formulations for showering, washing, bathing are also obtained if, instead of the polymer according to Example 3, one or more of the polymers 1, 2 or 4 to 12 are used.

1. A cosmetic composition comprising polymers which comprise, in copolymerized form, at least one compound of the general formula (I) and/or its cationic form (Ia) 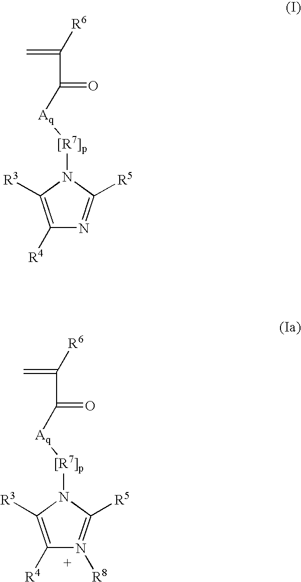
wherein
R³, R⁴ and R⁵, independently of one another, are H or C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₃-C₁₅-cycloalkylcarbonyl, aryl, arylcarbonyl, a heterocycle or a halogen atom;
R⁶ is H or methyl;
R⁷ is a divalent organic radical;
R⁸ is H or C₁-C₁₀-alkyl;
A is O or NH; and
p, q independently of one another are 0 or 1.

2. The cosmetic preparation according to claim 1, wherein the compound of the general formula (I) is 2-(1-imidazolyl)ethyl methacrylate and/or the compound of the general formula (Ia) is 2-(1-imidazolyl)ethyl methacrylate quaternized on the nitrogen.

3. The cosmetic preparation according to claim 1, wherein the polymers comprise at least one further monomer in copolymerized form.

4. The cosmetic preparation according to claim 3, wherein the at least one further monomer is selected from N-vinyllactams, monomers comprising acid groups, (meth)acrylic acid esters, (meth)acrylamides, vinyl ethers and diallylamines.

5. The where cosmetic preparation according to claim 3, wherein the at least one further monomer is selected from N-vinylpyrrolidone, (meth)acrylic acid, methyl methacrylate, N,N-diallyl-N,N-dimethylammonium chloride, N,N-dimethylamino ethyl methacrylate, N-[3-(dimethyl-amino)propyl]methacrylamide, methacrylamide and mixtures thereof.

6. The cosmetic preparation according to claim 1, wherein the polymers comprise at least two further monomers in copolymerized form, where at least one of the further monomers is selected from N-vinyllactams and at least one of the further monomers is selected from (meth)acrylamides.

7. (canceled)

8. (canceled)

9. (canceled)

10. (canceled)

11. The cosmetic preparation according to claim 2, wherein the polymers comprise at least one further monomer in copolymerized form.

12. The cosmetic preparation according to claim 4, wherein the at least one further monomer is selected from N-vinylpyrrolidone, (meth)acrylic acid, methyl methacrylate, N,N-diallyl-N,N-dimethylammonium chloride, N,N-dimethylaminoethyl methacrylate, N-[3-(dimethylamino)propyl]methacrylamide, methacrylamide and mixtures thereof.

13. A conditioner comprising the cosmetic preparation according to claim 1.

14. A hair-setting agent comprising the cosmetic preparation according to claim 1.

15. A thickener comprising the cosmetic preparation according to claim 1.
